SEQUENCE LISTING (105 sequences)

SEQ ID NO: 1 - protein, 8 aa, synthetic construct
  REGION 1..8: note = CDR1 sequence of the heavy chain variable region
GFNFNTYT

SEQ ID NO: 2 - protein, 10 aa, synthetic construct
  REGION 1..10: note = CDR1 sequence of the heavy chain variable region
GGSISSINYY

SEQ ID NO: 3 - protein, 10 aa, synthetic construct
  REGION 1..10: note = CDR1 sequence of the heavy chain variable region
GDSVSRRSVA

SEQ ID NO: 4 - protein, 10 aa, synthetic construct
  REGION 1..10: note = CDR1 sequence of the heavy chain variable region
GGSISSRNFY

SEQ ID NO: 5 - protein, 8 aa, synthetic construct
  REGION 1..8: note = CDR2 sequence of the heavy chain variable region
ISSSSSYI

SEQ ID NO: 6 - protein, 7 aa, synthetic construct
  REGION 1..7: note = CDR2 sequence of the heavy chain variable region
INYSGTT

SEQ ID NO: 7 - protein, 9 aa, synthetic construct
  REGION 1..9: note = CDR2 sequence of the heavy chain variable region
TYYRSKWFS

SEQ ID NO: 8 - protein, 7 aa, synthetic construct
  REGION 1..7: note = CDR2 sequence of the heavy chain variable region
IYTSGST

SEQ ID NO: 9 - protein, 25 aa, synthetic construct
  REGION 1..25: note = CDR3 sequence of the heavy chain variable region
TRVNPQAKGSDWLDPPINQYYGMDV

SEQ ID NO: 10 - protein, 20 aa, synthetic construct
  REGION 1..20: note = CDR3 sequence of the heavy chain variable region
ARQTYYYDRRGYYRPEPIEH

SEQ ID NO: 11 - protein, 18 aa, synthetic construct
  REGION 1..18: note = CDR3 sequence of the heavy chain variable region
RKSKGRQQLAESTSSVWT

SEQ ID NO: 12 - protein, 20 aa, synthetic construct
  REGION 1..20: note = CDR3 sequence of the heavy chain variable region
ARGTFYYDRSGNGRLDPLDY

SEQ ID NO: 13 - protein, 7 aa, synthetic construct
  REGION 1..7: note = CDR1 sequence of the light chain variable region
QSVSSSY

SEQ ID NO: 14 - protein, 6 aa, synthetic construct
  REGION 1..6: note = CDR1 sequence of the light chain variable region
QDVRSF

SEQ ID NO: 15 - protein, 6 aa, synthetic construct
  REGION 1..6: note = CDR1 sequence of the light chain variable region
QGIRND

SEQ ID NO: 16 - protein, 6 aa, synthetic construct
  REGION 1..6: note = CDR1 sequence of the light chain variable region
QDISNY

SEQ ID NO: 17 - DNA, 22 bp, synthetic construct
  misc_feature 1..22: note = Forward primer for the amplification of the heavy chain VH135
  source 1..22: mol_type = other DNA
aggtgcagctgctcgagtctgg

SEQ ID NO: 18 - DNA, 29 bp, synthetic construct
  misc_feature 1..29: note = Reverse primer or the amplification of heavy chain of IgG1
  source 1..29: mol_type = other DNA
gcatgtactagttttgtcacaagatttgg

SEQ ID NO: 19 - DNA, 23 bp, synthetic construct
  misc_feature 1..23: note = Forward primer for the amplification of VH2
  source 1..23: mol_type = other DNA
cagatcaccttgctcgagtctgg

SEQ ID NO: 20 - protein, 9 aa, synthetic construct
  REGION 1..9: note = CDR3 sequence of the light chain variable region
QQYGSSPWT

SEQ ID NO: 21 - protein, 5 aa, synthetic construct
  REGION 1..5: note = CDR3 sequence of the light chain variable region
QQYVT

SEQ ID NO: 22 - protein, 9 aa, synthetic construct
  REGION 1..9: note = CDR3 sequence of the light chain variable region
QQTYDTPLT

SEQ ID NO: 23 - protein, 9 aa, synthetic construct
  REGION 1..9: note = CDR3 sequence of the light chain variable region
LQDYNFPRT

SEQ ID NO: 24 - protein, 8 aa, synthetic construct
  REGION 1..8: note = CDR3 sequence of the light chain variable region
QQHDNLVT

SEQ ID NO: 25 - protein, 21 aa, synthetic construct
  REGION 1..21: note = FR1 sequence of the heavy chain variable region
LESGGGLVKPGGSLRLSCAAS

SEQ ID NO: 26 - protein, 21 aa, unidentified
  REGION 1..21: note = FR1 sequence of the heavy chain variable region
LEWGPGLVKPSETLSLTCTVS

SEQ ID NO: 27 - protein, 21 aa, synthetic construct
  REGION 1..21: note = FR1 sequence of the heavy chain variable sequence
LESGPGLLKPSQSLSLTCAIS

SEQ ID NO: 28 - protein, 21 aa, synthetic construct
  REGION 1..21: note = FR1 sequence of the heavy chain variable region
LEWGPGLVKASQTLSLTCTVS

SEQ ID NO: 29 - protein, 17 aa, synthetic construct
  REGION 1..17: note = FR2 sequence of the heavy chain variable region
MNWVRQAPGKGLEWVSS

SEQ ID NO: 30 - protein, 17 aa, synthetic construct
  REGION 1..17: note = FR2 sequence of the heavy chain variable region
WVWIRQPPGKGLEWIGN

SEQ ID NO: 31 - protein, 17 aa, synthetic construct
  REGION 1..17: note = FR2 sequence of the heavy chain variable region
WNWIRQSPSRGLEWLGR

SEQ ID NO: 32 - protein, 17 aa, synthetic construct
  REGION 1..17: note = FR2 sequence of the heavy chain variable region
WSWIRQPGGKGLEWIGR

SEQ ID NO: 33 - protein, 38 aa, synthetic construct
  REGION 1..38: note = FR3 sequence of the heavy chain variable region
DNADSVKGRFTIYRDNAKKSLYLRMIGLRVEDSGVYYC

SEQ ID NO: 34 - protein, 38 aa, synthetic construct
  REGION 1..38: note = FR3 sequence of the heavy chain variable region
NYNPSLKSRVTISVDTSKNQFSLKLSSVTAADTAVYYC

SEQ ID NO: 35 - protein, 38 aa, synthetic construct
  REGION 1..38: note = FR3 sequence of the heavy chain variable region
EYGVSVRGRITISPDTTKNQFSLQLNSVTPEDTAVYYC

SEQ ID NO: 36 - protein, 38 aa, synthetic construct
  REGION 1..38: note = FR3 sequence of the heavy chain variable region
NYNPSLKSRVTISLDTSKSQFSLKLSSVTAADTAVYYC

SEQ ID NO: 37 - protein, 11 aa, synthetic construct
  REGION 1..11: note = FR4 sequence of the heavy chain variable region
WGQGTTVTVSS

SEQ ID NO: 38 - protein, 11 aa, synthetic construct
  REGION 1..11: note = FR4 sequence of the heavy chain variable region
WGQRDHGHRSP

SEQ ID NO: 39 - protein, 11 aa, synthetic construct
  REGION 1..11: note = FR4 sequence of the heavy chain variable region
WGQGTLVTVSS

SEQ ID NO: 40 - protein, 11 aa, synthetic construct
  REGION 1..11: note = FR4 sequence of the heavy chain variable region
WGQGTTVIVYS

SEQ ID NO: 41 - protein, 26 aa, synthetic construct
  REGION 1..26: note = FR1 sequence of the light chain variable region
ELTLTQSPGTLSLSPGERATLSCRAS

SEQ ID NO: 42 - protein, 26 aa, synthetic construct
  REGION 1..26: note = FR1 sequence of the light chain variable region
ELTLTQSPATLSLSPGDRATLSCRAS

SEQ ID NO: 43 - protein, 26 aa, synthetic construct
  REGION 1..26: note = FR1 sequence of the light chain variable region
ELVMTQSPSFLSASVGDRVTITCRAS

SEQ ID NO: 44 - protein, 26 aa, synthetic construct
  REGION 1..26: note = FR1 sequence of the light chain variable region
ELVMTQSPSSLSAFVGDRVTLTCRAS

SEQ ID NO: 45 - protein, 26 aa, synthetic construct
  REGION 1..26: note = FR1 sequence of the light chain variable region
ELVMTQSPSSLSASVGDRVTITCQAS

SEQ ID NO: 46 - protein, 17 aa, synthetic construct
  REGION 1..17: note = FR2 sequence of the light chain variable region
LAWYQQKPGQAPRLLIY

SEQ ID NO: 47 - protein, 17 aa, synthetic construct
  REGION 1..17: note = FR2 sequence of the light chain variable region
LAWYQHKPGQPPRLLIY

SEQ ID NO: 48 - protein, 17 aa, synthetic construct
  REGION 1..17: note = FR2 sequence of the light chain variable region
LHWYQQRPGKAPKLLIY

SEQ ID NO: 49 - protein, 17 aa, synthetic construct
  REGION 1..17: note = FR2 sequence of the light chain variable region
LNWYQQKPGQPPKLLIY

SEQ ID NO: 50 - protein, 17 aa, synthetic construct
  REGION 1..17: note = FR2 sequence of the light chain variable region
LNWYQQKPGKAPKLLIY

SEQ ID NO: 51 - protein, 36 aa, synthetic construct [sequence omitted]

SEQ ID NO: 52 - protein, 36 aa, synthetic construct
  REGION 1..36: note = FR3 sequence of the light chain variable region
SRAAGIPDRFSGSGSGTDFTLTISRLEPEDFAVYYC

SEQ ID NO: 53 - protein, 36 aa, synthetic construct
  REGION 1..36: note = FR3 sequence of the light chain variable region
MVSSEVPSRFSGSGSETDFTLTIDGLQPEDVATYFC

SEQ ID NO: 54 - protein, 36 aa, synthetic construct
  REGION 1..36: note = FR3 sequence of the light chain variable region
ALQSGVPSRFSGSGFGTDFTLTISSLQPEDIATYYC

SEQ ID NO: 55 - protein, 36 aa, synthetic construct
  REGION 1..36: note = FR3 sequence of the light chain variable region
NLETGVPSRFSGSGFGTHFTLTISSLQPEDFATYYC

SEQ ID NO: 56 - protein, 10 aa, synthetic construct
  REGION 1..10: note = FR4 sequence of the light chain variable region
FGQGTKVEIK

SEQ ID NO: 57 - protein, 10 aa, synthetic construct
  REGION 1..10: note = FR4 sequence of the light chain variable region
FGPGTKVDIK

SEQ ID NO: 58 - protein, 10 aa, synthetic construct
  REGION 1..10: note = FR4 sequence of the light chain variable region
FGGGTAVDIK

SEQ ID NO: 59 - protein, 10 aa, synthetic construct
  REGION 1..10: note = FR4 sequence of the light chain variable region
FGGGTKVEIK

SEQ ID NO: 60 - protein, 128 aa, synthetic construct
  REGION 1..128: note = Fab#5 heavy chain variable sequence
LESGGGLVKPGGSLRLSCAASGFNFNTYTMNWVRQAPGKGLEWVSSISSSSSYIDNADSVKGRFTIYRDNAKKSLYLRMIGLRVEDSGVYYCTRVNPQAKGSDWLDPPINQYYGMDVWGQGTTVTVSS

SEQ ID NO: 61 - protein, 108 aa, synthetic construct
  REGION 1..108: note = Fab#5 light chain variable sequence
ELTLTQSPGTLSLSPGERATLSCRASQSVSSSYLAWYQQKPGQAPRLLIYGASSRATGIPDRFSGSGSGTDFTLTISRLEPEDFAVYYCQQYGSSPWTFGQGTKVEIK

SEQ ID NO: 62 - protein, 128 aa, synthetic construct
  REGION 1..128: note = BU2 heavy chain variable sequence
LESGGGLVKPGGSLRLSCAASGFNFNTYTMNWVRQAPGKGLEWVSSISSSSSYIDNADSVKGRFTIYRDNAKKSLYLRMIGLRVEDSGVYYCTRVNPQAKGSDWLDPPINQYYGMDVWGQRDHGHRSP

SEQ ID NO: 63 - protein, 104 aa, synthetic construct
  REGION 1..104: note = BU2 light chain variable sequence
ELTLTQSPATLSLSPGDRATLSCRASQSVSSSYLAWYQHKPGQPPRLLIYGASSRAAGIPDRFSGSGSGTDFTLTISRLEPEDFAVYYCQQYVTFGPGTKVDIK

SEQ ID NO: 64 - protein, 124 aa, synthetic construct
  REGION 1..124: note = BU7 heavy chain variable sequence
LEWGPGLVKPSETLSLTCTVSGGSISSINYYWVWIRQPPGKGLEWIGNINYSGTTNYNPSLKSRVTISVDTSKNQFSLKLSSVTAADTAVYYCARQTYYYDRRGYYRPEPIEHWGQGTLVTVSS

SEQ ID NO: 65 - protein, 107 aa, synthetic construct
  REGION 1..107: note = BU7 light chain variable sequence
ELVMTQSPSFLSASVGDRVTITCRASQDVRSFLHWYQQRPGKAPKLLIYAASMVSSEVPSRFSGSGSETDFTLTIDGLQPEDVATYFCQQTYDTPLTFGGGTAVDIK

SEQ ID NO: 66 - protein, 124 aa, synthetic construct
  REGION 1..124: note = BU11 heavy chain variable sequence
LESGPGLLKPSQSLSLTCAISGDSVSRRSVAWNWIRQSPSRGLEWLGRTYYRSKWFSEYGVSVRGRITISPDTTKNQFSLQLNSVTPEDTAVYYCRKSKGRQQLAESTSSVWTWGQGTTVIVYS

SEQ ID NO: 67 - protein, 107 aa, synthetic construct
  REGION 1..107: note = BU11 light chain variable sequence
ELVMTQSPSSLSAFVGDRVTLTCRASQGIRNDLNWYQQKPGQPPKLLIYAASALQSGVPSRFSGSGFGTDFTLTISSLQPEDIATYYCLQDYNFPRTFGQGTKVEIK

SEQ ID NO: 68 - protein, 123 aa, synthetic construct
  REGION 1..123: note = BU54 heavy chain variable sequence
LEWGPGLVKASQTLSLTCTVSGGSISSRNFYWSWIRQPGGKGLEWIGRIYTSGSTNYNPSLKSRVTISLDTSKSQFSLKLSSVTAADTAVYYCARGTFYYDRSGNGRLDPLDYWGQGTLVTVS

SEQ ID NO: 69 - protein, 106 aa, synthetic construct
  REGION 1..106: note = BU54 light chain variable sequence
ELVMTQSPSSLSASVGDRVTITCQASQDISNYLNWYQQKPGKAPKLLIYDASNLETGVPSRFSGSGFGTHFTLTISSLQPEDFATYYCQQHDNLVTFGGGTKVEIK

SEQ ID NO: 70 - protein, 120 aa, synthetic construct
  REGION 1..120: note = Amino acid sequence of the heavy chain variable sequence of FabBS.70
LESGGGVVQPGTSLRLSCAASGFTFNNFGMHWVRQAPGKGLEWVAMISYEGSKDFYADSVKGRFTISKDHARNTVYLQMNSLRAEDTAEYYCAKDKAIFMISAGRTLDFWGQGTLVTVSS

SEQ ID NO: 71 - protein, 107 aa, synthetic construct
  REGION 1..107: note = Amino acid sequence of a light chain variable sequence of FabBS.70
ELVMTQSPSSLSASVGDRVTITCRASQNIGIYLNWYQQKPGKAPKLLIYAASSLQNGVPSRFSGSGSGTDFTLTISTLQPEDFATYWCQQGYSTPLTFGGGTKVEIR

SEQ ID NO: 72 - DNA, 384 bp, synthetic construct
  misc_feature 1..384: note = DNA sequence encoding for the heavy chain variable region ofclone Fab#5
  source 1..384: mol_type = other DNA
ctcgagtctgggggaggcctggtcaagcctggggggtccctgagactctcctgtgcagcctctggattcaacttcaatacctataccatgaactgggtccgccaggctccagggaaggggctggagtgggtctcatccattagtagtagtagtagttacatagacaacgcagactcagtgaagggccgattcaccatctacagagacaacgccaagaagtcactgtatctgcgaatgatcggcctgagagtcgaggactccggtgtgtattactgtacgcgagtgaacccccaggccaaagggtcggactggttggatccccccatcaaccaatactacggtatggacgtctggggccaagggaccacggtcaccgtctcctca

SEQ ID NO: 73 - DNA, 324 bp, synthetic construct
  misc_feature 1..324: note = DNA sequence encoding for the light chain variable region ofclone Fab #5
  source 1..324: mol_type = other DNA
gagctcacactcacgcagtctccaggcaccctgtctttgtctccaggggaaagagccaccctctcctgcagggccagtcagagtgttagcagcagctatttagcctggtaccagcagaaacctggccaggctcccaggctcctcatctatggtgcatccagcagggccactggcatcccagacaggttcagtggcagtgggtctgggacagacttcactctcaccatcagcagactggagcctgaagattttgcagtgtattactgtcagcagtatggtagctcaccgtggacgttcggccaagggaccaaggttgaaatcaaa

SEQ ID NO: 74 - DNA, 384 bp, synthetic construct
  misc_feature 1..384: note = DNA sequence encoding for the heavy chain variable region ofclone Fab BU.2
  source 1..384: mol_type = other DNA
ctcgagtctgggggaggcctggtcaagcctggggggtccctgagactctcctgtgcagcctctggattcaacttcaatacctataccatgaactgggtccgccaggctccagggaaggggctggagtgggtctcatccattagtagtagtagtagttacatagacaacgcagactcagtgaagggccgattcaccatctacagagacaacgccaagaagtcactgtatctgcgaatgatcggcctgagagtcgaggactccggtgtgtattactgtacgcgagtgaacccccaggccaaagggtcggactggttggatccccccatcaaccaatactacggtatggacgtctggggccaaagggaccacggtcaccggtctcct

SEQ ID NO: 75 - DNA, 312 bp, synthetic construct
  misc_feature 1..312: note = DNA sequence encoding for the light chain variable region ofclone Fab BU.2
  source 1..312: mol_type = other DNA
gagctcacactcacgcagtctccagccaccctgtctttgtctccaggggatagagccaccctctcctgcagggccagtcagagtgttagcagcagttacttagcctggtaccagcacaaacctggccagccccccaggctcctcatctatggtgcatccagcagggccgccggcatcccagacaggttcagtggcagtgggtctgggacagacttcactctcaccatcagcagactggagcctgaagattttgcagtgtattactgtcagcagtacgtcactttcggccctgggaccaaagtggatatcaaa

SEQ ID NO: 76 - DNA, 372 bp, synthetic construct
  misc_feature 1..372: note = DNA sequence encoding for the heavy chain variable region ofclone Fab BU.7
  source 1..372: mol_type = other DNA
ctcgagtggggcccaggactggtgaagccttcggagaccctgtccctcacctgcactgtctctggtggctccatcagcagcataaattactactgggtctggattcgccagcccccagggaaggggctggagtggattgggaatatcaattacagtgggaccaccaactacaacccgtccctcaagagtcgagtcaccatatccgtggacacgtccaagaaccagttctccctgaagctgagctctgtgaccgccgcagacacggctgtctattactgtgcgagacaaacgtattactatgataggcgtggttattaccgcccggagcccattgagcactggggccagggaaccctggtcaccgtctcctca

SEQ ID NO: 77 - DNA, 321 bp, synthetic construct
  misc_feature 1..321: note = DNA sequence encoding for the light chain variable region ofclone Fab BU.7
  source 1..321: mol_type = other DNA
gagctcgtgatgacacagtctccatccttcctgtctgcatctgtaggagacagagtcaccatcacttgccgggcaagtcaagatgttagaagttttttacattggtatcaacaaagaccagggaaagcccctaagttgttgatctatgctgcatccatggtgtcgagtgaggtcccgtcaaggttcagtggcagtggatctgagacagatttcactctcaccatcgacggtctgcaacctgaagatgttgcaacttacttctgtcaacagacttacgacacgccgctcaccttcggcggcgggaccgcggttgacatcaaa

SEQ ID NO: 78 - DNA, 372 bp, synthetic construct
  misc_feature 1..372: note = DNA sequence encoding for the heavy chain variable region ofclone Fab BU.11
  source 1..372: mol_type = other DNA
ctcgagtcaggtccaggactgctgaagccctcgcagagcctctcactcacctgtgccatctccggagacagtgtctctaggagaagtgttgcttggaactggatcagacagtccccatcgagaggccttgagtggctgggaaggacatactacaggtccaagtggttttctgagtatggcgtatctgtgagaggtcgaataaccatcagtccagacacaaccaagaaccagttctccctgcagctgaactccgtgactcccgaggacacggctgtctattactgtcgaaagtcgaaagggaggcagcagttggcagagtctacgagttcggtatggacgtggggccaagggaccacggtcatcgtctactca

SEQ ID NO: 79 - DNA, 321 bp, synthetic construct
  misc_feature 1..321: note = DNA sequence encoding for the light chain variable region ofclone Fab BU.11
  source 1..321: mol_type = other DNA
gagctcgtgatgacacagtctccatcctccctgtctgcatttgtgggagatagagtcaccctcacttgccgggcaagtcagggcattagaaatgatttaaactggtatcagcagaaaccagggcaaccccctaaactcctgatctatgctgcatccgctttacaaagtggcgtcccatcaaggttcagcggcagtgggtttggcacagatttcactctcaccatcagcagcctgcagcctgaagatattgcaacttattactgtctacaagattacaatttccctcggacgttcggccaagggaccaaggtggaaatcaaa

SEQ ID NO: 80 - DNA, 372 bp, synthetic construct
  misc_feature 1..372: note = DNA sequence encoding for the heavy chain variable region ofclone Fab BU.54
  source 1..372: mol_type = other DNA
ctcgagtggggcccaggactggtgaaggcttcacagaccctgtccctcacctgcactgtctctggtggctccatcagcagtaggaatttctattggagttggatccggcagcccggcgggaagggactggagtggattgggcgtatctatacaagtgggagcaccaattacaatccctccctcaagagtcgagtcactatatcattagacacgtccaagagtcagttctccctgaagctgagctctgtgaccgccgcagacacggccgtgtattattgtgcgagagggacgttttattatgataggagtggtaatggtcgattagatccgcttgactactggggccagggaaccctggtcaccgtctcctca

SEQ ID NO: 81 - DNA, 318 bp, synthetic construct
  misc_feature 1..318: note = DNA sequence encoding for the light chain variable region ofclone Fab BU.54
  source 1..318: mol_type = other DNA
gagctcgtgatgacacagtctccatcctccctgtctgcatctgtaggagacagagtcaccatcacttgccaggcgagtcaggacattagcaactatttaaattggtatcagcagaaaccagggaaagcccctaagctcctgatctacgatgcatccaatttggaaacaggggtcccatcaaggttcagtggaagtggatttgggacacattttacgttaaccatcagcagcctgcagcctgaagattttgcaacatattactgtcaacagcatgataatctcgtcactttcggcggagggaccaaggtggagatcaaa

SEQ ID NO: 82 - DNA, 361 bp, synthetic construct
  misc_feature 1..361: note = DNA sequence encoding for the heavy chain variable region ofclone Fab BS.70
  source 1..361: mol_type = other DNA
ctcgagtctgggggaggcgtggtccagcctgggacgtccctgagactctcctgtgcggcctctggattcaccttcaataattttgggatgcactgggtccgccaggctccaggcaagggtctggagtgggtggcaatgatctcatatgaaggaagtaaggatttctatgcagactccgtgaagggccgattcaccatctccaaagaccatgccaggaatacggtctatctgcaaatgaacagcctgagagctgaggacacggcagaatattactgtgcgaaagataaggctatatttatgatttctgccggacggactttggacttctggggccagggaaccctggtcaccgtctcctcag

SEQ ID NO: 83 - DNA, 322 bp, synthetic construct
  misc_feature 1..322: note = DNA sequence encoding for the light chain variable region ofclone Fab BS.70
  source 1..322: mol_type = other DNA
gagctcgtgatgacacagtctccatcctccctgtctgcatctgtaggagacagagtcaccattacttgccgggcaagtcagaacattggcatctatttgaattggtatcagcagaaacctgggaaagccccaaagctcctgatctatgctgcatccagtttgcaaaatggggtcccatcgaggttcagtggcagtggatctgggacagacttcactctcaccatcagcactctgcaacctgaagattttgcaacttactggtgtcaacagggttacagtaccccactcactttcggcggagggaccaaggtagagatcagac

SEQ ID NO: 84 - DNA, 23 bp, synthetic construct
  misc_feature 1..23: note = Forward primer for the amplification of the heavy chain VH4
  source 1..23: mol_type = other DNA
caggtgcagctgctcgagtcggg

SEQ ID NO: 85 - DNA, 23 bp, synthetic construct
  misc_feature 1..23: note = Forward primer for the amplification of the heavy chain VH4b
  source 1..23: mol_type = other DNA
caggtgcagctactcgagtgggg

SEQ ID NO: 86 - DNA, 24 bp, synthetic construct
  misc_feature 1..24: note = Forward primer for the amplification of the heavy chain VH4gs
  source 1..24: mol_type = other DNA
caggtgcagctactcgagtggggc

SEQ ID NO: 87 - DNA, 23 bp, synthetic construct
  misc_feature 1..23: note = Forward primer for the amplification of the heavy chain VH6
  source 1..23: mol_type = other DNA
caggtacagctgctcgagtcagg

SEQ ID NO: 88 - DNA, 38 bp, synthetic construct
  misc_feature 1..38: note = Forward primer for the amplification of the light chain VK1
  source 1..38: mol_type = other DNA
gagccgcacgagcccgagctccagatgacccagtctcc

SEQ ID NO: 89 - DNA, 58 bp, synthetic construct
  misc_feature 1..58: note = Reverse primer for the amplification of CK1d
  source 1..58: mol_type = other DNA
gcgccgtctagaattaacactctcccctgttgaagctctttgtgacgggcgaactcag

SEQ ID NO: 90 - DNA, 38 bp, synthetic construct
  misc_feature 1..38: note = Forward primer for the amplification of VK3 light chain
  source 1..38: mol_type = other DNA
gagccgcacgagcccgagctcgtgatgacacagtctcc

SEQ ID NO: 91 - DNA, 38 bp, synthetic construct
  misc_feature 1..38: note = Forward primer for the amplification of the light chain VK2/4
  source 1..38: mol_type = other DNA
gagccgcacgagcccgagctcgtgatgacccagtctcc

SEQ ID NO: 92 - DNA, 38 bp, synthetic construct
  misc_feature 1..38: note = Forward primer for the amplification of the light chain VK5
  source 1..38: mol_type = other DNA
gagccgcacgagcccgagctcacactcacgcagtctcc

SEQ ID NO: 93 - protein, 8 aa, synthetic construct
  REGION 1..8: note = Heavy chain variable seqeunce of CDR1
GFTFNNFG

SEQ ID NO: 94 - protein, 8 aa, synthetic construct
  REGION 1..8: note = Heavy chain variable sequence CDR2
ISYEGSKD

SEQ ID NO: 95 - protein, 17 aa, synthetic construct
  REGION 1..17: note = Heavy chain variable sequence CDR3
AKDKAIFMISAGRTLDF

SEQ ID NO: 96 - protein, 6 aa, synthetic construct
  REGION 1..6: note = Light chain amino acid sequence CDR1
QNIGIY

SEQ ID NO: 97 - protein, 9 aa, synthetic construct
  REGION 1..9: note = Light chain amino acid sequence CDR3
QQGYSTPLT

SEQ ID NO: 98 - protein, 21 aa, synthetic construct
  REGION 1..21: note = Heavy chain aminoacid sequence FR1
LESGGGVVQPGTSLRLSCAAS

SEQ ID NO: 99 - protein, 17 aa, synthetic construct
  REGION 1..17: note = Heavy chain amino acid sequence FR2
MHWVRQAPGKGLEWVAM

SEQ ID NO: 100 - protein, 38 aa, synthetic construct
  REGION 1..38: note = Heavy chain amino acid sequence FR3
FYADSVKGRFTISKDHARNTVYLQMNSLRAEDTAEYYC

SEQ ID NO: 101 - protein, 11 aa, synthetic construct
  REGION 1..11: note = Heavy chain amino acid sequence FR4
WGQGTLVTVSS

SEQ ID NO: 102 - protein, 26 aa, synthetic construct
  REGION 1..26: note = Light chain amino acid sequence FR1
ELVMTQSPSSLSASVGDRVTITCRAS

SEQ ID NO: 103 - protein, 17 aa, synthetic construct
  REGION 1..17: note = Light chain amino acid sequence FR2
LNWYQQKPGKAPKLLIY

SEQ ID NO: 104 - protein, 36 aa, synthetic construct
  REGION 1..36: note = Light chain amino acid sequence FR3
SLQNGVPSRFSGSGSGTDFTLTISTLQPEDFATYWC

SEQ ID NO: 105 - protein, 10 aa, synthetic construct
  REGION 1..10: note = Light chain amino acid sequence FR4
FGGGTKVEIR

(19) United States
(12) Patent Application Publication
(10) Pub. No.: US 2026/0234220 A1
(43) Pub. Date: Aug. 13, 2026

(54) NEUTRALIZING MONOCLONAL ANTIBODIES AGAINST SARBECOVIRUSES

(71) Applicant: OSPEDALE SAN RAFFAELE S.R.L., Milano (IT)

(72) Inventors: Nicasio MANCINI, Cernusco sul Naviglio (MI) (IT); Nicola CLEMENTI, Senigallia (AN) (IT); Massimo CLEMENTI, Milano (IT); Elena CRISCUOLO, Segrate (MI) (IT); Sofia SISTI, Sora (FR) (IT); Martina LIBERA, Cermenate (CO) (IT)

(21) Appl. No.: 18/647,774

(22) PCT Filed: Jul. 20, 2022
(86) PCT No.: PCT/IB2022/056706

§ 371 Date: Apr. 26, 2024

(30) Foreign Application Priority Data
Oct. 29, 2021 (EP) 21205626.1

(51) Int. Cl. C07K 16/104; G01N 33/569

(52) U.S. Cl. CPC C07K 16/104; G01N 33/56983; C07K 2317/55; C07K 2317/565; C07K 2317/567; C07K 2317/76; C07K 2317/92

(57) ABSTRACT

The present invention provides neutralizing monoclonal antibodies targeting the spike protein of Sarbecoviruses, such as Severe Acute Respiratory Syndrome-Coronavirus-2 (SARS-CoV-1 or SARS-CoV-2 like COVID-19). The monoclonal antibodies of the invention can inhibit or neutralize SARS-CoV-1 or SARS-CoV-2 activity and advantageously be used for treating, preventing or diagnosing COVID-19 infection in humans due to their significant cross-reactivity among SARS-CoV-2 variants.

### REFERENCE TO AN ELECTRONIC SEQUENCE LISTING

The contents of the electronic sequence listing “2048334RevSequenceListing040626.xml”, comprising 141,039 bytes and created on Apr. 6, 2026, is herein incorporated by reference in its entirety.

### BACKGROUND

Coronaviridae family comprises a large number of virus species that are further classified in four genera. Seven species are of human interest and are highly diversified in terms of adaptation, pathogenicity and diffusion. Indeed, four species are endemic and highly adapted to humans (HCoV-229E, HCoV-NL63, HCoV-OC43 and HCoV-HKU1), two are epidemic (MERS-CoV and the extinct SARS-CoV) and one pandemic, the recently emerged and currently circulating SARS-CoV and SARS-CoV-2 belong to the same genus (Betacoronavirus) and subgenus (Sarbecovirus) but are phylogenetically distinct and have strikingly different features in terms of infectivity and clinical signs. These aspects demonstrate the difficulties in thoroughly sampling the entire Coronaviridae family necessary to implement an effective surveillance program and therefore stress the need to have therapeutic strategies with broad activity on CoVs.

Commonly, the ideal target to inhibit a virus replication is represented by the proteins that mediate entry because this prevents cell infection. Entry inhibitors can thus be used as effective therapeutics and, whether endowed with long half-life, in prophylaxis. The relevance of inhibiting virus entry is demonstrated by the fact that most neutralizing antibodies elicited by natural infection or vaccination recognizes fundamental regions of the viral proteins involved in entry.

Also, the long half-life of monoclonal antibodies (mAbs) allows their use in prophylaxis. While immunization will be the most effective approach to limit and possibly eradicate SARS-CoV-2, the identification of neutralizing mAbs (nAbs) will be fundamental to address vaccines intrinsic limitations regarding the delayed response, the fraction of non-responders and the impossibility to vaccinate immunocompromised individuals. Also, there are several open questions regarding a long-lasting humoral immunity against SARS-CoV-2 and the effect of variants continuously emerging that may partially hamper vaccination efficacy in the long run. Altogether, neutralizing mAbs against SARS-CoV-2 are and will be a fundamental therapeutic and prophylactic tool and the availability of a panel of diverse mAbs will help coping with the emergence of new variants. Furthermore, since a few mAbs naturally cross-neutralize SARS-CoV and SARS-CoV-2 and recently an in vitro matured mAb (named ADG-2) neutralizing several Sarbecoviruses has been described, there is the possibility to identify cross-reactive mAbs.

The entire entry process of all CoVs into host cells is mediated by the spike (S), a large transmembrane glycoprotein protruding from the virus envelope. The spike is a homotrimer in which each monomer is produced as a single protein (S0) that is cleaved by host proteases into the S1 and S2 subunits. S1 is located apically and contains the receptor binding domain (RBD), therefore it mediates the attachment phase by interacting with host proteins. S2 forms the spike stalk that contains the fusion peptide, the transmembrane domain, and the heptad repeats (HR1 and HR2), all necessary to fuse the virus envelope to the host membranes.

CoVs entry is a multistep process that involves receptor engagement of all three RBDs in each spike trimer, the proteolytic cleavage of at least one specific spike site and the subsequent fusion.

SARS-CoV-2, in analogy with SARS-CoV and several animal CoVs, specifically binds to the angiotensin-converting enzyme 2 (ACE2). The binding event is necessary but not sufficient to mediate entry, as SARS-CoV-2 S requires to be processed by host proteases that also specifically determine the virus entry site. Indeed, if the spike is cleaved by furin and TMPRSS2, SARS-CoV-2 fusion happens at the plasma membrane, conversely it enters from the endosomal compartment, where the spike is cleaved by cathepsins. Once the spike is completely engaged and cleaved, S1 dissociates from S2 allowing the exposure of the fusion peptide and its insertion in the host membranes. The final molecular event leading to fusion is a dramatic conformational change of S2 that involves the entire subunit and is driven by the refolding of HR1 and HR2. Notably, all events from binding to fusion require significant structural rearrangements and therefore can potentially be the target of neutralizing nAbs.

The spike protein of CoVs is the most prominent protein exposed on virions surface and the main target of the humoral response.

Characterization of sera from SARS-CoV-2 infected individuals has identified several neutralizing epitopes located on the spike protein that either overlap with domains fundamental for its function or indirectly inhibit the conformational changes required to mediate attachment and fusion.

Most of the neutralizing epitopes identified so far are conformational and located in the RBD; all nAbs binding to it can be grouped in four classes, characterized by different modes of action. Class 1 and class 2 antibodies directly hamper the interaction between ACE2 and the spike by recognizing the same subdomain in the RBD, the so-called receptor binding motif (RBM), while class 3 and 4 bind cryptic epitopes that indirectly block the correct RBM exposure or the downstream conformational changes required for fusion.

The broadly neutralizing mAb ADG-2, albeit with a unique binding mode, recognizes an epitope partially shared with class 1 nAbs, highlighting how even minor differences in terms of residues involved and angle of approach can dramatically impact the biologic activity of a nAb. At a lower frequency, nAbs recognizing other epitopes have been identified as well. Indeed, strong neutralization has been documented to a mAb that binds to a region (N-terminal domain-NTD) in S1 not directly involved in entry.

Also, the presence in patient sera of neutralizing antibodies recognizing linear epitopes located downstream of the RBD or overlapping the fusion peptide has been identified. See Table 1 of Lanying Du et al., 2021 [1] for reference, summarizing representative human neutralizing monoclonal antibodies (nAbs) against SARS-CoV-2 and the relative target antigens (i.e. RBD, NTD, etc.).

Altogether, while the neutralizing response in patient sera is dominated by antibodies specific for the RBD, several other spike domains can be effectively targeted. Worth mentioning, the RBD, and S1 in general, are the spike most variable domains, both considering SARS-CoV-2 variants continuously emerging and among CoVs belonging to the same sub-genus.

Therefore, there is an urgent need of and extreme interest for nAbs targeting conserved spike protein domains, as they reasonably have a broader spectrum of neutralizable isolates/species.

Therapeutic mAbs for COVID-19 treatment have been developed in accelerated time and the pace has been unprecedented for any disease.

Currently, 8 SARS-CoV-2 RBD-specific potent nAbs have been approved by the Food and Drug Administration (FDA) under an emergency use authorization (EUA) to treat COVID-19 non-hospitalized patients at high risk of severe illness.

The following COVID-19 mAbs are in clinical use: bamlanivimab (LY-CoV555) [2]; a combination of bamlanivimab (LY-CoV555) and etesevimab (LY-CoV016 or JS016) [3] from Eli Lilly; a combination of casirivimab (REGN10933) and Imdevimab (REGN10987) [4] from Regeneron (REGEN-COV); a combination cilgavimab (COV2-2130 or AZD1061) and tixagevimab (COV2-2196 or AZD8955) [5] from AstraZeneca; monotherapy-based nAbs sotrovimab (VIR-7831) [6] from GSK and Vir Biotechnology; and regdanvimab (CT-P59) [7] from Celltrion. Another set of monotherapy and combination Nabs-based therapies are under Phase III trials: 2B04 [8] and 47D11 [9] from AbbVie; BRII-196 and BRII-198 from Brii Biosciences [10]; and TY027 from Tychan are also in Phase III trials [10].

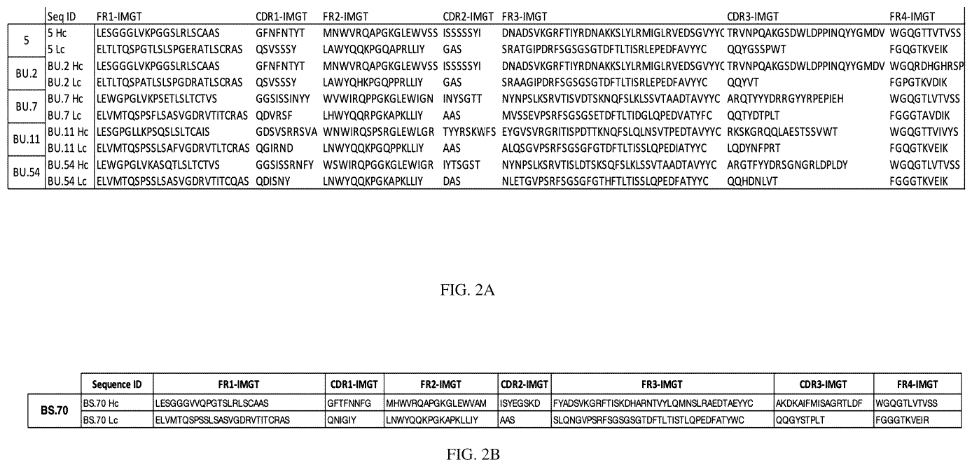

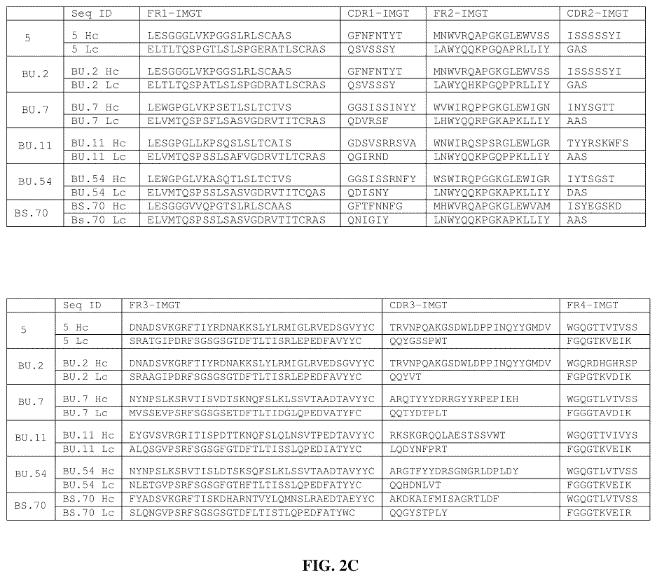

A comprehensive list of nAbs that are currently in Phase I, II, and III trials and in clinic is summarized in FIG. 2A of Kumar S. et al., 2021 [11], herein included as reference.

Several SARS-CoV-2 variants are being reported from different parts of the world. According to the World Health Organization (WHO), a recognized mutation is elevated to a “variant of concern” (VOC) when the acquisition of a new mutation allows for increased viral transmission, increased fatality, and a significant decrease in the effectiveness of therapy and vaccines. A “variant of interest” (VOI) is a variant with a new mutation capable of affecting disease severity, transmissibility, immune and diagnostic escape.

The current variants of concern are Alpha (B.1.1.7, identified in the United Kingdom) [12], Beta (B.1.351, identified in South Africa) [13], Gamma (P.1, identified in Brazil) [14], Delta (B.1.617.2, identified in India) and Omicron sublineages and descendent lineages (Omicron (B.1.1.529) [16], Omicron BA.2 (B.1.1.529+BA.2) Omicron BA.4 (B.1.1.529+BA.4) and Omicron BA.5.2 (B.1.1.529+BA.5)). Current variants of interest are Eta (B.1.525, identified in UK/Nigeria), Iota (B.1.526, identified in the United States of America) [17], Kappa (B.1.617.1, identified in India) [15], and Lambda (C.37, identified in Peru) [18]. See for reference FIG. 2B of Kumar S. et al., 2021 providing an exhaustive list of mutations present in the current SARS-Cov-2 VOCs and VOIs.

Ideally, an effective antiviral therapeutic strategy should have the ability to prevent infection/disease by new variants while simultaneously maintaining breadth against existing multiple viral strains/variants. Recent studies have reported that many NTD-specific NAbs are relatively less effective to all emerging variants, whereas RBD-specific NAbs are variably effective against emerging variants and VOCs. The majority of the potent therapeutic nAbs as monotherapy showed complete abrogation or reduced neutralizing activity against SARS-CoV-2 emerging variants that contain the E484K/Q or L452R mutations.

For example, Bamlanivimab (LY-CoV555) was ineffective against all VOCs and thus was no longer considered for EUA.

Currently, combination therapies comprising a cocktail of nAbs targeting distinct nonoverlapping epitopes on RBD have demonstrated exceptional potency and promising correlates of protection against SARS-CoV-2 and its variants. See for reference Additionally, newly identified RBD core-binding nAbs SARS2-38 and LY-CoV1404 as monotherapy potently neutralize all variants of concern of SARS-CoV-2.

The authors of the present invention have now identified neutralizing antibodies targeting conserved regions of the spike protein of SARS-CoV-1 and SARS-CoV-2 as promising and attractive therapeutic candidates to be used alone or in combination to tackle the disease burden caused by SARS-CoV-2 in all the VOCs and VOIs known sofar.

Therefore, it is an object of the present invention an isolated antibody or antibody fragment binding a conserved region of the spike protein of Sarbecoviruses, characterized by an antigen binding site comprising:
a) a heavy chain variable sequence comprising:
   i) a CDR1 comprising an amino acid sequence selected from the group consisting of SEQ ID NO:1, SEQ ID NO:2, SEQ ID NO:3, SEQ ID NO:4 and SEQ ID NO: 93;
   ii) a CDR2 comprising an amino acid sequence selected from the group consisting of SEQ ID NO:5, SEQ ID NO:6, SEQ ID NO:7, SEQ ID NO:8 and SEQ ID NO:94;
   iii) a CDR3 comprising an amino acid sequence selected from the group consisting of SEQ ID NO:9, SEQ ID NO:10, SEQ ID NO:11, SEQ ID NO:12 and SEQ ID NO:95;
      and/or
b) a light chain variable sequence comprising:
   i) a CDR1 comprising an amino acid sequence selected from the group consisting of SEQ ID NO:13, SEQ ID NO:14, SEQ ID NO:15, SEQ ID NO:16 and SEQ ID NO:96;
   ii) a CDR2 comprising an amino acid sequence selected from the group consisting of GAS, AAS and DAS;
   iii) a CDR3 comprising an amino acid sequence selected from the group consisting of SEQ ID NO:20, SEQ ID NO:21, SEQ ID NO:22, SEQ ID NO:23, SEQ ID NO:24 and SEQ ID NO:97.

Complementary determining regions (CDRs; CDR1-CDR3) of the variable domain of heavy and constant regions and framework regions (FRs; FR1-FR4) characterizing the sequence of the isolated antibody or antibody fragment of the invention have been determined according to IMGT nomenclature.

According to preferred embodiment the heavy chain variable sequence the antibody or antibody fragment of the invention, further comprises:
a) a framework region (FR1) comprising an amino acid sequence selected from the group consisting of SEQ ID NO:25, SEQ ID NO:26, SEQ ID NO:27, SEQ ID NO:28 and SEQ ID NO:98;
b) a framework region (FR2) comprising an amino acid sequence selected from the group consisting of SEQ ID NO:29, SEQ ID NO:30, SEQ ID NO:31, SEQ ID NO:32 and SEQ ID NO:99;
c) a framework region (FR3) comprising an amino acid sequence selected from the group consisting of SEQ ID NO:33, SEQ ID NO:34, SEQ ID NO:35, SEQ ID NO:36 and SEQ ID NO:100;
d) a framework region (FR4) comprising an amino acid sequence selected from the group consisting of SEQ ID NO:37, SEQ ID NO:38, SEQ ID NO:39, SEQ ID NO:40, and SEQ ID NO:101;
and/or
wherein the light chain variable sequence further comprises:
a) a framework region (FR1) comprising an amino acid sequence selected from the group consisting of SEQ ID NO:41, SEQ ID NO:42, SEQ ID NO:43; SEQ ID NO:44, SEQ ID NO:45 and SEQ ID NO:102;
b) a framework region (FR2) comprising an amino acid sequence selected from the group consisting of SEQ ID NO:46, SEQ ID NO:47, SEQ ID NO:48, SEQ ID NO:49, SEQ ID NO:50 and SEQ ID NO:103;
c) a framework region (FR3) comprising an amino acid sequence selected from the group consisting of SEQ ID NO:51, SEQ ID NO:52, SEQ ID NO:53, SEQ ID NO:54, SEQ ID NO:55 and SEQ ID NO:104;
d) a framework region (FR4) comprising an amino acid sequence selected from the group consisting of SEQ ID NO:56, SEQ ID NO:57, SEQ ID NO:58, SEQ ID NO:59 and SEQ ID NO:105.

According to preferred embodiments of the present invention the antibody or antibody fragment is characterized by an antigen binding site comprising:
a) a heavy chain variable sequence comprising a CDR1 comprising the amino acid sequence SEQ ID NO:1; a CDR2 comprising the amino acid sequence SEQ ID NO:5; a CDR3 comprising the amino acid sequence SEQ ID NO:9 and/or a light chain variable sequence comprising a CDR1 comprising the amino acid sequence SEQ ID NO:13; a CDR2 comprising the amino acid sequence GAS; a CDR3 comprising the amino acid sequence SEQ ID NO:20. According to another preferred embodiment said heavy chain variable sequence may further comprises: a framework region FR1 comprising SEQ ID NO:25; a framework region FR2 comprising SEQ ID NO:29; a framework region FR3 comprising SEQ ID NO:33 and a framework region FR4 comprising SEQ ID NO:37; said light chain variable sequence may further comprises a framework region FR1 comprising SEQ ID NO:41, a framework region FR2 comprising SEQ ID NO:46, a framework region FR3 comprising SEQ ID NO:51, and a framework region FR4 comprising SEQ ID NO:56;
b) a heavy chain variable sequence comprising: a CDR1 comprising the amino acid sequence SEQ ID NO:1; a CDR2 comprising the amino acid sequence SEQ ID NO:5; a CDR3 comprising the amino acid sequence SEQ ID NO:9 and/or a light chain variable sequence comprising a CDR1 comprising the amino acid sequence SEQ ID NO:13; a CDR2 comprising the amino acid sequence GAS; a CDR3 comprising the amino acid sequence SEQ ID NO:21. According to another preferred embodiment said heavy chain variable sequence may further comprises a framework region FR1 comprising SEQ ID NO:25; a framework region FR2 comprising SEQ ID NO:29; a framework region FR3 comprising SEQ ID NO:33 and a framework region FR4 comprising SEQ ID NO:38; said light chain variable sequence may further comprises a framework region FR1 comprising SEQ ID NO:42; a framework region FR2 comprising SEQ ID NO:47; a framework region FR3 comprising SEQ ID NO:52, and a framework region FR4 comprising SEQ ID NO:57;
c) a heavy chain variable sequence comprising a CDR1 comprising the amino acid sequence SEQ ID NO:2; a CDR2 comprising the amino acid sequence SEQ ID NO:6; a CDR3 comprising the amino acid sequence SEQ ID NO:10 and/or a light chain variable sequence comprising a CDR1 comprising the amino acid sequence SEQ ID NO:14; a CDR2 comprising the amino acid sequence AAS; a CDR3 comprising the amino acid sequence SEQ ID NO:22. According to another preferred embodiment said heavy chain variable sequence may further comprises a framework region FR1 consisting of SEQ ID NO:26; a framework region FR2 consisting of SEQ ID NO:30 a framework region FR3 comprising SEQ ID NO:34, and a framework region FR4 comprising SEQ ID NO:39; said light chain variable sequence may further comprises a framework region FR1 comprising SEQ ID NO:43; a framework region FR2 comprising SEQ ID NO:48; a framework region FR3 comprising SEQ ID NO:53 and a framework region FR4 comprising SEQ ID NO:58;
d) a heavy chain variable sequence comprising a CDR1 comprising the amino acid sequence SEQ ID NO:3; a CDR2 comprising the amino acid sequence SEQ ID NO:7; a CDR3 comprising the amino acid sequence SEQ ID NO:11 and/or a light chain variable sequence comprising a CDR1 comprising the amino acid sequence SEQ ID NO:15; a CDR2 comprising the amino acid sequence AAS; a CDR3 comprising the amino acid sequence SEQ ID NO:23. According to another preferred embodiment said heavy chain variable sequence may further comprises a framework region FR1 comprising SEQ ID NO:27; a framework region FR2 comprising SEQ ID NO:31 a framework region FR3 comprising SEQ ID NO:35 and a framework region FR4 consisting of SEQ ID NO:40; said light chain variable sequence may further comprises a framework region FR1 comprising SEQ ID NO:44; a framework region FR2 comprising SEQ ID NO:49; a framework region FR3 comprising SEQ ID NO:54 and a framework region FR4 comprising SEQ ID NO:56;
e) a heavy chain variable sequence comprising a CDR1 comprising the amino acid sequence SEQ ID NO:4; a CDR2 comprising the amino acid sequence SEQ ID NO:8; a CDR3 comprising the amino acid sequence SEQ ID NO:12; and/or a light chain variable sequence comprising: a CDR1 comprising the amino acid sequence SEQ ID NO:16; a CDR2 comprising the amino acid sequence DAS; a CDR3 comprising the amino acid sequence SEQ ID NO:24. According to another preferred embodiment said heavy chain variable sequence may further comprises a framework region FR1 comprising SEQ ID NO:28; a framework region FR2 comprising SEQ ID NO:32; a framework region FR3 comprising SEQ ID NO:36 and a framework region FR4 comprising SEQ ID NO:39; said light chain variable sequence may further comprises a framework region FR1 comprising SEQ ID NO:45; a framework region FR2 comprising SEQ ID NO:50; a framework region FR3 comprising SEQ ID NO:55; and a framework region FR4 comprising SEQ ID NO:59;
f) a heavy chain variable sequence comprising a CDR1 comprising the amino acid sequence SEQ ID NO:93; a CDR2 comprising the amino acid sequence SEQ ID NO:94; a CDR3 comprising the amino acid sequence SEQ ID NO:95; and/or a light chain variable sequence comprising: a CDR1 comprising the amino acid sequence SEQ ID NO:96; a CDR2 comprising the amino acid sequence AAS; a CDR3 comprising the amino acid sequence SEQ ID NO:97. According to another preferred embodiment said heavy chain variable sequence may further comprises a framework region FR1 comprising SEQ ID NO:98; a framework region FR2 comprising SEQ ID NO:99; a framework region FR3 comprising SEQ ID NO:100 and a framework region FR4 comprising SEQ ID NO:101; said light chain variable sequence may further comprises a framework region FR1 comprising SEQ ID NO:102; a framework region FR2 comprising SEQ ID NO:103; a framework region FR3 comprising SEQ ID NO:104; and a framework region FR4 comprising SEQ ID NO:105.

According to a preferred embodiment of the invention the antibody or antibody fragment (clone Fab 5 #) of the invention comprises an heavy chain variable sequence comprising the amino acid sequence:

(SEQ ID NO: 60)
LESGGGLVKPGGSLRLSCAASGFNFNTYTMNWVRQAPGKGLEWVSS
ISSSSSYIDNADSVKGRFTIYRDNAKKSLYLRMIGLRVEDSGVYYC
TRVNPQAKGSDWLDPPINQYYGMDVWGQGTTVTVSS

and/or a light chain variable sequence comprising the amino acid sequence:

(SEQ ID NO: 61)
ELTLTQSPGTLSLSPGERATLSCRASQSVSSSYLAWYQQKPGQAPR
LLIYGASSRATGIPDRFSGSGSGTDFTLTISRLEPEDFAVYYCQQY
GSSPWTFGQGTKVEIK.

In another preferred embodiment the antibody or antibody fragment (clone Fab BU.2) of the invention comprises an heavy chain variable sequence comprising the amino acid sequence:

(SEQ ID NO: 62)
LESGGGLVKPGGSLRLSCAASGFNFNTYTMNWVRQAPGKGLEWVSS
ISSSSSYIDNADSVKGRFTIYRDNAKKSLYLRMIGLRVEDSGVYYC
TRVNPQAKGSDWLDPPINQYYGMDVWGQRDHGHRSP

and a light chain variable sequence comprising the amino acid sequence:

(SEQ ID NO: 63)
ELTLTQSPATLSLSPGDRATLSCRASQSVSSSYLAWYQHKPGQPPR
LLIYGASSRAAGIPDRFSGSGSGTDFTLTISRLEPEDFAVYYCQQY
VTFGPGTKVDIK

According to another preferred alternative embodiment of the invention the isolated antibody or antibody fragment (clone Fab BU.7) of the invention comprises an heavy chain variable sequence comprising the amino acid sequence:

(SEQ ID NO: 64)
LEWGPGLVKPSETLSLTCTVSGGSISSINYYWVWIRQPPGKGLEWI
GNINYSGTTNYNPSLKSRVTISVDTSKNQFSLKLSSVTAADTAVYY
CARQTYYYDRRGYYRPEPIEHWGQGTLVTVSS

and/or a light chain variable sequence comprising the amino acid sequence:

(SEQ ID NO: 65)
ELVMTQSPSFLSASVGDRVTITCRASQDVRSFLHWYQQRPGKAPKL
LIYAASMVSSEVPSRFSGSGSETDFTLTIDGLQPEDVATYFCQQTY
DTPLTFGGGTAVDIK.

In another embodiment of the invention the isolated antibody or antibody fragment of the invention (clone Fab BU.11) comprises an heavy chain variable sequence comprising the amino acid sequence:

(SEQ ID NO: 66)
LESGPGLLKPSQSLSLTCAISGDSVSRRSVAWNWIRQSPSRGLEWL
GRTYYRSKWFSEYGVSVRGRITISPDTTKNQFSLQLNSVTPEDTAV
YYCRKSKGRQQLAESTSSVWTWGQGTTVIVYS

and/or a light chain variable sequence comprising the amino acid sequence:

(SEQ ID NO: 67)
ELVMTQSPSSLSAFVGDRVTLTCRASQGIRNDLNWYQQKPGQPPKL
LIYAASALQSGVPSRFSGSGFGTDFTLTISSLQPEDIATYYCLQDY
NFPRTFGQGTKVEIK.

Still in another alternative preferred embodiment of the invention the isolated antibody or antibody fragment of the invention (clone Fab BU.54) comprises an heavy chain variable sequence comprising the amino acid sequence:

(SEQ ID NO: 68)
LEWGPGLVKASQTLSLTCTVSGGSISSRNFYWSWIRQPGGKGLEWI
GRIYTSGSTNYNPSLKSRVTISLDTSKSQFSLKLSSVTAADTAVYY
CARGTFYYDRSGNGRLDPLDYWGQGTLVTVS

and/or a light chain variable sequence comprising the amino acid sequence:

(SEQ ID NO: 69)
ELVMTQSPSSLSASVGDRVTITCQASQDISNYLNWYQQKPGKAPKL
LIYDASNLETGVPSRFSGSGFGTHFTLTISSLQPEDFATYYCQQHD
NLVTFGGGTKVEIK.

In a last preferred alternative embodiment of the invention the isolated antibody or antibody fragment (clone Fab BS.70) of the invention comprises an heavy chain variable sequence comprising the amino acid sequence:

(SEQ ID NO: 70)
LESGGGVVQPGTSLRLSCAASGFTFNNFGMHWVRQAPGKGLEWVAM
ISYEGSKDFYADSVKGRFTISKDHARNTVYLQMNSLRAEDTAEYYC
AKDKAIFMISAGRTLDFWGQGTLVTVSS

and a light chain variable sequence comprising the amino acid sequence:

(SEQ ID NO: 71)
ELVMTQSPSSLSASVGDRVTITCRASQNIGIYLNWYQQKPGKAPKL
LIYAASSLQNGVPSRFSGSGSGTDFTLTISTLQPEDFATYWCQQGY
STPLTFGGGTKVEIR

It is a further object of present invention a nucleotide sequence encoding for the heavy chain variable region and/or the light chain variable region of the antibody or antibody fragment of the invention above listed.

In a preferred embodiment the nucleotide sequences of the invention are selected from the group consisting of:

Heavy chain (clone Fab#5):
(SEQ ID NO: 72)
ctcgagtctgggggaggcctggtcaagcctggggggtccctgaga
ctctcctgtgcagcctctggattcaacttcaatacctataccatg
aactgggtccgccaggctccagggaaggggctggagtgggtctca
tccattagtagtagtagtagttacatagacaacgcagactcagtg
aagggccgattcaccatctacagagacaacgccaagaagtcactg
tatctgcgaatgatcggcctgagagtcgaggactccggtgtgtat
tactgtacgcgagtgaacccccaggccaaagggtcggactggttg
gatccccccatcaaccaatactacggtatggacgtctggggccaa
gggaccacggtcaccgtctcctca
Light chain (Fab#5):
(SEQ ID NO: 73)
gagctcacactcacgcagtctccaggcaccctgtctttgtctcca
ggggaaagagccaccctctcctgcagggccagtcagagtgttagc
agcagctatttagcctggtaccagcagaaacctggccaggctccc
aggctcctcatctatggtgcatccagcagggccactggcatccca
gacaggttcagtggcagtgggtctgggacagacttcactctcacc
atcagcagactggagcctgaagattttgcagtgtattactgtcag
cagtatggtagctcaccgtggacgttcggccaagggaccaaggtt
gaaatcaaa
Heavy chain (Fab BU.2)
(SEQ ID NO: 74)
ctcgagtctgggggaggcctggtcaagcctggggggtccctgaga
ctctcctgtgcagcctctggattcaacttcaatacctataccatg
aactgggtccgccaggctccagggaaggggctggagtgggtctca
tccattagtagtagtagtagttacatagacaacgcagactcagtg
aagggccgattcaccatctacagagacaacgccaagaagtcactg
tatctgcgaatgatcggcctgagagtcgaggactccggtgtgtat
tactgtacgcgagtgaacccccaggccaaagggtcggactggttg
gatccccccatcaaccaatactacggtatggacgtctggggccaa
agggaccacggtcaccggtctcct
Light chain (Fab BU.2)
(SEQ ID NO: 75)
gagctcacactcacgcagtctccagccaccctgtctttgtctcca
ggggatagagccaccctctcctgcagggccagtcagagtgttagc
agcagttacttagcctggtaccagcacaaacctggccagcccccc
aggctcctcatctatggtgcatccagcagggccgccggcatccca
gacaggttcagtggcagtgggtctgggacagacttcactctcacc
atcagcagactggagcctgaagattttgcagtgtattactgtcag
cagtacgtcactttcggccctgggaccaaagtggatatcaaa
Heavy chain (Fab BU.7)
(SEQ ID NO: 76)
ctcgagtggggcccaggactggtgaagccttcggagaccctgtcc
ctcacctgcactgtctctggtggctccatcagcagcataaattac
tactgggtctggattcgccagcccccagggaaggggctggagtgg
attgggaatatcaattacagtgggaccaccaactacaacccgtcc
ctcaagagtcgagtcaccatatccgtggacacgtccaagaaccag
ttctccctgaagctgagctctgtgaccgccgcagacacggctgtc
tattactgtgcgagacaaacgtattactatgataggcgtggttat
taccgcccggagcccattgagcactggggccagggaaccctggtc
accgtctcctca
Light chain (Fab BU.7):
(SEQ ID NO: 77)
gagctcgtgatgacacagtctccatccttcctgtctgcatctgta
ggagacagagtcaccatcacttgccgggcaagtcaagatgttaga
agttttttacattggtatcaacaaagaccagggaaagcccctaag
ttgttgatctatgctgcatccatggtgtcgagtgaggtcccgtca
aggttcagtggcagtggatctgagacagatttcactctcaccatc
gacggtctgcaacctgaagatgttgcaacttacttctgtcaacag
acttacgacacgccgctcaccttcggcggcgggaccgcggttgac
atcaaa
Heavy chain (Fab BU.11)
(SEQ ID NO: 78)
ctcgagtcaggtccaggactgctgaagccctcgcagagcctctca
ctcacctgtgccatctccggagacagtgtctctaggagaagtgtt
gcttggaactggatcagacagtccccatcgagaggccttgagtgg
ctgggaaggacatactacaggtccaagtggttttctgagtatggc
gtatctgtgagaggtcgaataaccatcagtccagacacaaccaag
aaccagttctccctgcagctgaactccgtgactcccgaggacacg
gctgtctattactgtcgaaagtcgaaagggaggcagcagttggca
gagtctacgagttcggtatggacgtggggccaagggaccacggtc
atcgtctactca
Light chain (Fab BU.11)
(SEQ ID NO: 79)
gagctcgtgatgacacagtctccatcctccctgtctgcatttgtg
ggagatagagtcaccctcacttgccgggcaagtcagggcattaga
aatgatttaaactggtatcagcagaaaccagggcaaccccctaaa
ctcctgatctatgctgcatccgctttacaaagtggcgtcccatca
aggttcagcggcagtgggtttggcacagatttcactctcaccatc
agcagcctgcagcctgaagatattgcaacttattactgtctacaa
gattacaatttccctcggacgttcggccaagggaccaaggtggaa
atcaaa
Heavy chain (Fab BU.54)
(SEQ ID NO: 80)
ctcgagtggggcccaggactggtgaaggcttcacagaccctgtcc
ctcacctgcactgtctctggtggctccatcagcagtaggaatttc
tattggagttggatccggcagcccggcgggaagggactggagtgg
attgggcgtatctatacaagtgggagcaccaattacaatccctcc
ctcaagagtcgagtcactatatcattagacacgtccaagagtcag
ttctccctgaagctgagctctgtgaccgccgcagacacggccgtg
tattattgtgcgagagggacgttttattatgataggagtggtaat
ggtcgattagatccgcttgactactggggccagggaaccctggtc
accgtctcctca
Light chain (Fab BU.54)
(SEQ ID NO: 81)
gagctcgtgatgacacagtctccatcctccctgtctgcatctgta
ggagacagagtcaccatcacttgccaggcgagtcaggacattagc
aactatttaaattggtatcagcagaaaccagggaaagcccctaag
ctcctgatctacgatgcatccaatttggaaacaggggtcccatca
aggttcagtggaagtggatttgggacacattttacgttaaccatc
agcagcctgcagcctgaagattttgcaacatattactgtcaacag
catgataatctcgtcactttcggcggagggaccaaggtggagatc
aaa
Heavy chain (BS.70):
(SEQ ID NO: 82)
ctcgagtctgggggaggcgtggtccagcctgggacgtccctgaga
ctctcctgtgcggcctctggattcaccttcaataattttgggatg
cactgggtccgccaggctccaggcaagggtctggagtgggtggca
atgatctcatatgaaggaagtaaggatttctatgcagactccgtg
aagggccgattcaccatctccaaagaccatgccaggaatacggtc
tatctgcaaatgaacagcctgagagctgaggacacggcagaatat
tactgtgcgaaagataaggctatatttatgatttctgccggacgg
actttggacttctggggccagggaaccctggtcaccgtctcctca
g
Light chain (Fab BS.70):
(SEQ ID NO: 83)
gagctcgtgatgacacagtctccatcctccctgtctgcatctgta
ggagacagagtcaccattacttgccgggcaagtcagaacattggc
atctatttgaattggtatcagcagaaacctgggaaagccccaaag
ctcctgatctatgctgcatccagtttgcaaaatggggtcccatcg
aggttcagtggcagtggatctgggacagacttcactctcaccatc
agcactctgcaacctgaagattttgcaacttactggtgtcaacag
ggttacagtaccccactcactttcggcggagggaccaaggtagag
atcagac.

Additionally, the invention relates to an expression vector comprising at least one of the nucleotide sequence encoding for the heavy chain and/or the light chain variable region of the antibody or antibody fragment of the invention.

A further object of the invention contemplates an host cell comprising the vector above outlined.

It is another object of the invention an hybridoma comprising at least one of the nucleotide sequence encoding for the heavy chain and/or the light chain variable region of the antibody or antibody fragment of the invention.

Additionally, the invention contemplates a formulation for molecular/vector-based passive immunoprophylaxis comprising at least one of the nucleotide sequence or expression vector of the invention together with one or more pharmaceutically acceptable excipients and/or adjuvants. The vaccine formulation may be advantageously be used in the prevention of Sarbecoviruses-mediated diseases in a subject, in particular of the Severe Acute Respiratory Syndrome mediated by SARS-CoV-1 or SARS-CoV-2.

Preferably the antibody or antibody fragment of the invention is a monoclonal antibody. In another preferred embodiment the antibody fragment is a Fab fragment. Even more preferably the antibody or antibody fragment of the invention is a human antibody.

Preferably, the antibody or antibody fragment is a human IgG or an IgG fragment selected from the group consisting of IgG1, IgG2, IgG3, IgG4 or a recombinant IgG comprising a mutated Fc portion to improve their binding and neutralization activity or all the Fc-mediated immune functions.

In alternative embodiments the antibody or antibody fragment of the invention are in a format selected among the group comprising single chain antibodies (scFv), nanobodies, bispecific antibodies, monoclonal antibodies conjugated with drugs and/or molecules influencing their pharmacokinetics and/or their delivery.

According to a further embodiment the antibody or antibody fragment of the invention is labelled with a marker or conjugated with a drug.

As an example, antitumour activity has been accomplished by conjugating antibodies with different effector molecules that accomplish cell death after antibody binding and internalisation. Such effector molecules include cytotoxic agents [19, 20], bacterial or plant protein toxins (immunotoxins) [21, 22], and radiopharmaceutical agents [23].

A further object of the present invention is the use of the antibody or antibody fragment of the invention to detect the presence of a Sarbecovirus, preferably of SARS-CoV-1 or SARS-CoV-2 in a biological sample.

The present invention further contemplates the antibody or antibody fragment of the invention for use in medical field for the treatment and/or prophylaxis of Sarbecoviruses-mediated diseases in a subject, in particular of the Severe Acute Respiratory Syndrome mediated by SARS-CoV-1 or SARS-CoV-2.

The term “SARS-Cov-2” encompasses anyone of the variants of concern (VOCs) Alpha (B.1.1.7), Beta (B.1.351) Gamma (P.1), Delta (B.1.617.2), D614 G (Università di Pavia) and Omicron (B.1.1.529) or of the variants of interest (VOIs) Eta (B.1.525), Iota (B.1.526), Kappa (B.1.617.1) and Lambda (C.37), known sofar.

The antibody or antibody fragment of the present invention may be used alone or in combination with another antibody or antibody fragment directed against different conserved regions of the spike protein. The combined use of the antibody or antibody fragment of the invention enhances the cross reactivity of the therapeutic treatment towards the different VOCs of SARS-CoV-2. The second antibody may be also a different antibody with respect to the claimed ones. Combinations of antibodies targeting different epitopes can translate into enhanced therapeutic efficacy, i.e., overcoming drug-related side effects of already approved molecules or clearing drug resistance of possible novel circulating variants.

According to a preferred embodiment the subject is an immunocompromised patient or a patient with an history of cardiovascular and/or respiratory diseases. Alternatively the patient may be an elderly patient (that is older than 70 years) or a vaccine-hesitant subject.

The antibody or antibody fragment according to the invention may be advantageously therapeutically administered in a human being also in a combination antibody cocktail.

Preferably, the antibody or antibody fragment of the invention is administered after 5-10 days from occurrence of SARS-CoV-1 or SARS-CoV-2 infection.

It is an object of the present invention a pharmaceutical composition comprising at least one of the antibody or antibody fragment of the invention as the active ingredient, together with one or more pharmaceutically acceptable excipients and/or adjuvants. In a preferred embodiment the antibody or antibody fragment is adapted to systemic administration by intravenous route. The invention alternatively contemplates intramuscular or subcutaneous administration route.

The present invention further relates to a pharmaceutical composition comprising at least one of the antibody or antibody fragment of the invention for use in the treatment or prophylaxis of Sarbecoviruses-mediated diseases in a subject, in particular of the Severe Acute Respiratory Syndrome mediated by SARS-CoV-1 or SARS-CoV-2. It is a further object of the present invention a composition or kit of parts comprising a first antibody or antibody fragment that binds a targeted conserved region of the spike protein of SARS-CoV-1 or SARS-CoV-2 of the invention and a second antibody or antibody fragment that binds a different targeted conserved region of the spike protein of SARS CoV-1 or SARS-CoV-2, for the simultaneous, separate or sequential administration in a subject affected by Sarbecoviruses-mediated diseases, in particular by the Severe Acute Respiratory Syndrome mediated by SARS CoV-1 or SARS-CoV-2. Preferably the first antibody or antibody fragment and the second antibody or antibody fragment are in separate containers.

The present invention will now be described, for non-limiting illustrative purposes, according to preferred embodiments thereof, with particular reference to the attached figures, wherein:

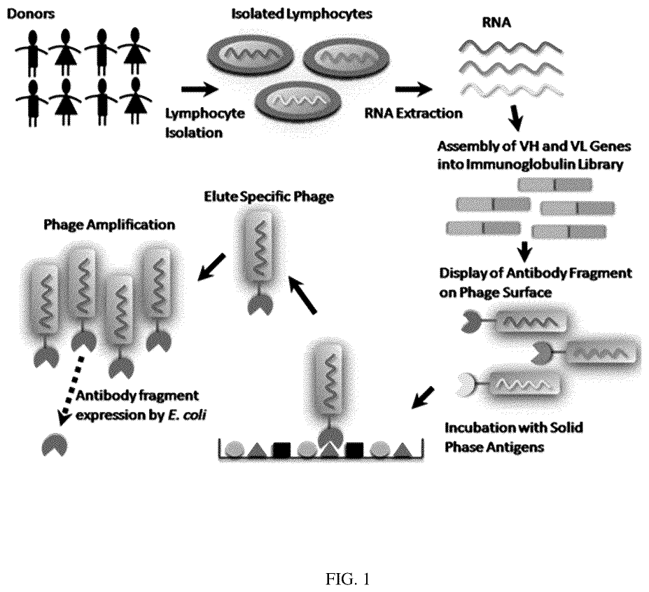

FIG. 1 shows a general scheme for the preparation and selection of a naïve recombinant antibody library. This figure indicates the general steps involved in the construction and selection of a recombinant antibody from a phage display library. The B-lymphocytes are first isolated from the blood of the donors. The total mRNA is extracted from the isolated lymphocyte cells and reverse-transcribed to complementary DNA (cDNA). The variable heavy (VH) and variable light (VL) chain antibody genes are amplified by polymerase chain reaction with gene-specific primers and assembled before cloning into a phage display vector. The resulting phages display antibody fragments on their surfaces. During selection, filamentous phage display libraries are incubated with antigens immobilized on a solid phase. The antigen-binding phages remains attached to the antigen, whereas the unbound phages are washed away. The bound phages are then eluted and amplified by infection with bacterial cells. By changing the expression host to a non-suppressor bacterial strain, the soluble antibody fragment can be expressed and secreted to the culture medium.

FIG. 2 shows the whole amino acid (panel A) and nucleotide sequences (panel B) of the heavy chain variable region and of the light chain variable region for each clone, specifying both the framework regions FR1-FR4 and the complementary determining regions CDR1-CDR3.

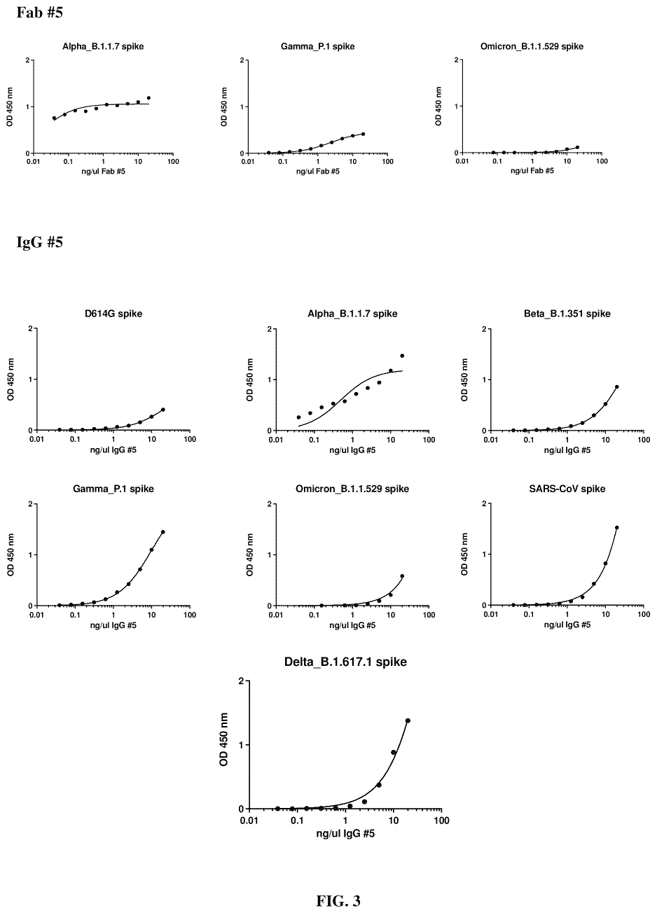

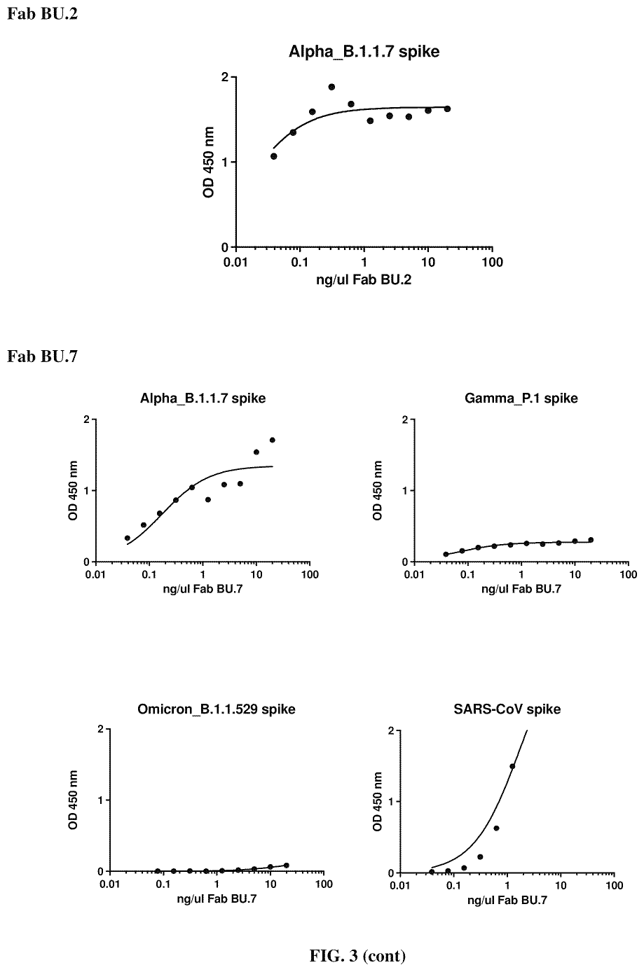

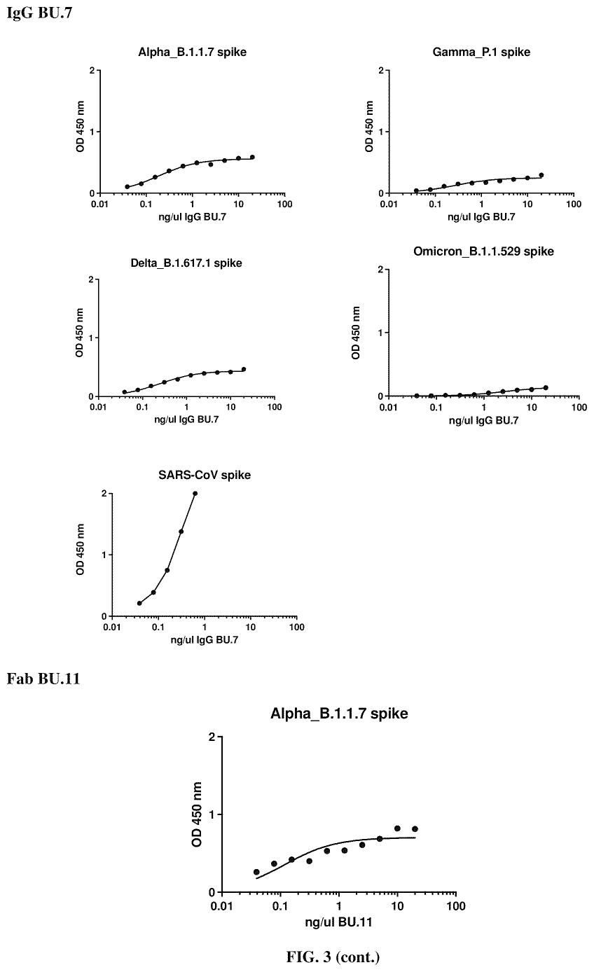

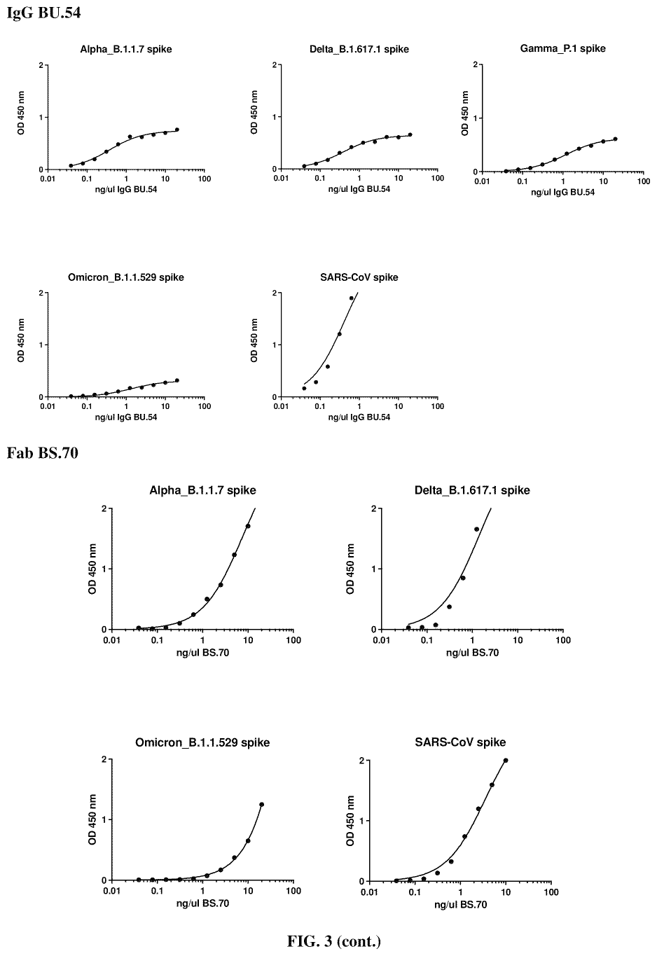

FIG. 3 shows the binding activity of the selected clones (#5, BU.2, BU.7, BU.11, BU.54, BS.70) both as Fab and IgG1 format assessed by ELISA assay. The selected Fabs and IgG were tested for their binding to the recombinant protein of the different VOCs.

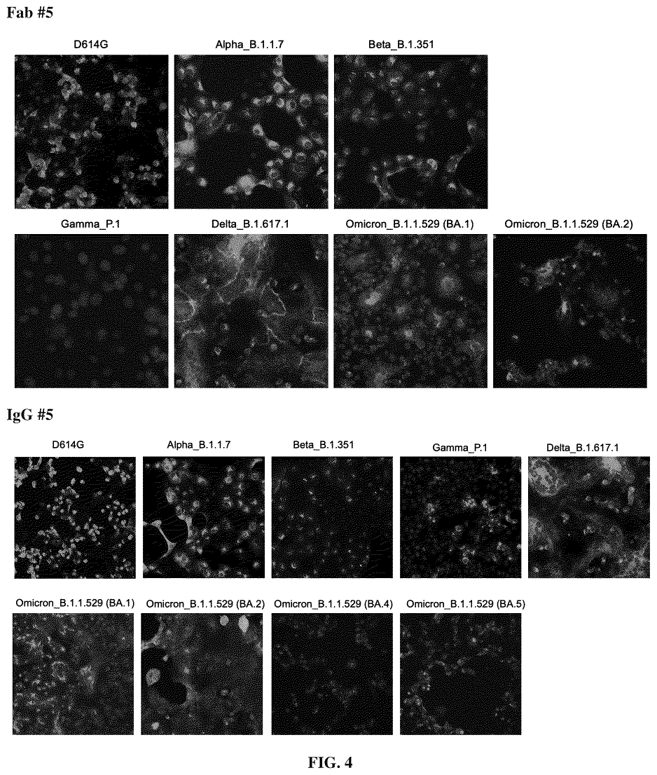

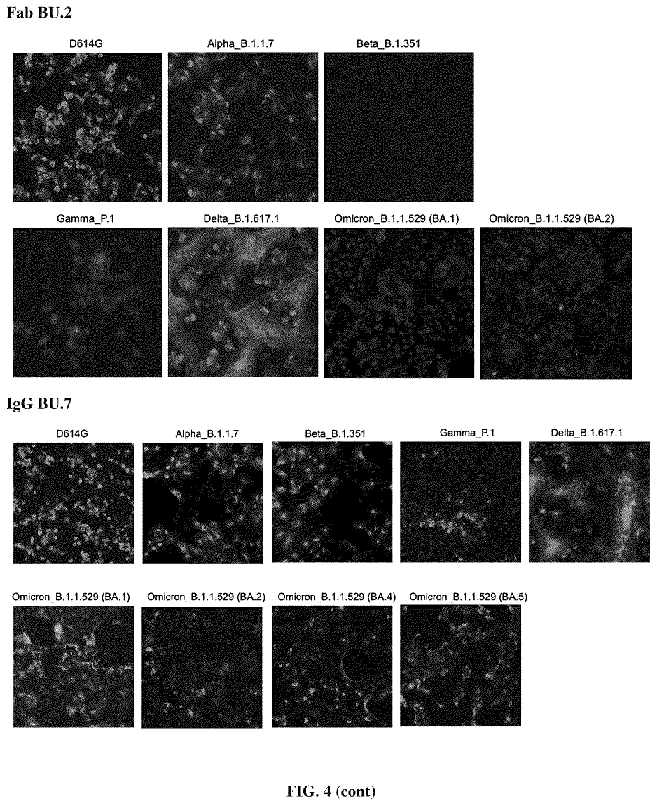

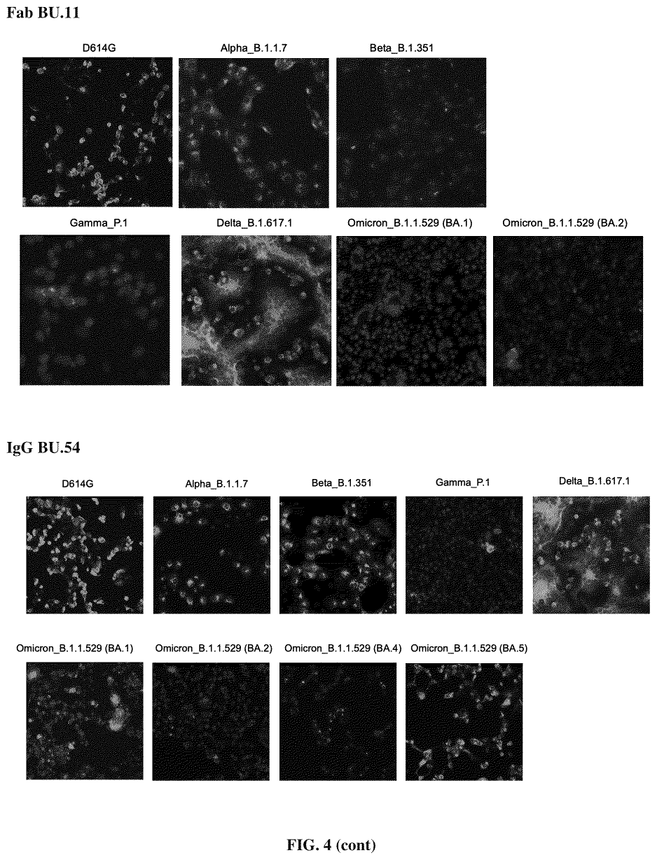

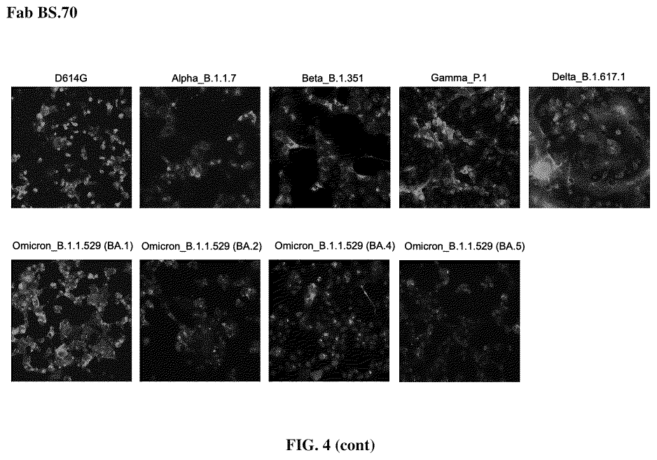

FIG. 4 shows the results of immunofluorescence analysis of the binding activity of the selected clones, both as Fab and IgG1 format. The selected Fabs can detect the VeroE6 cells infected by D614G, Alpha_B.1.1.7, Beta_B.1.135, Gamma P.1, Delta_B.1.617.1, Omicron_B.1.529 (BA.1), Omicron_B.1.529 (BA.2), Omicron_B.1.529 (BA.4) and Omicron_B.1.529 (BA.5) viral variants.

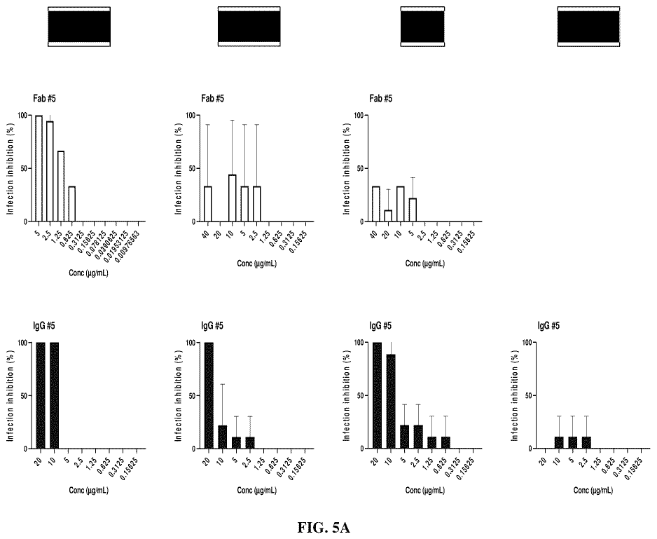

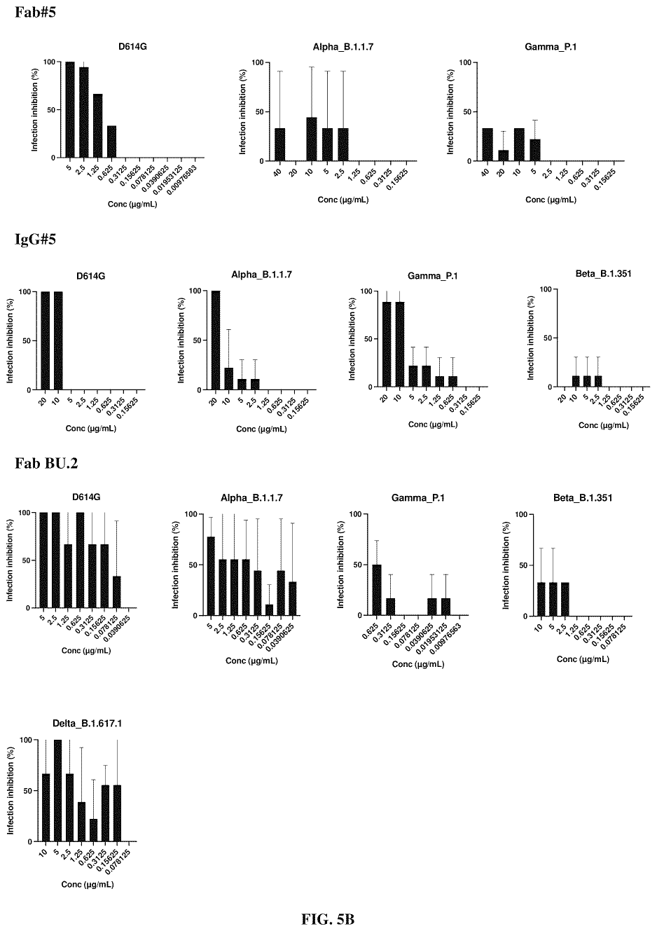

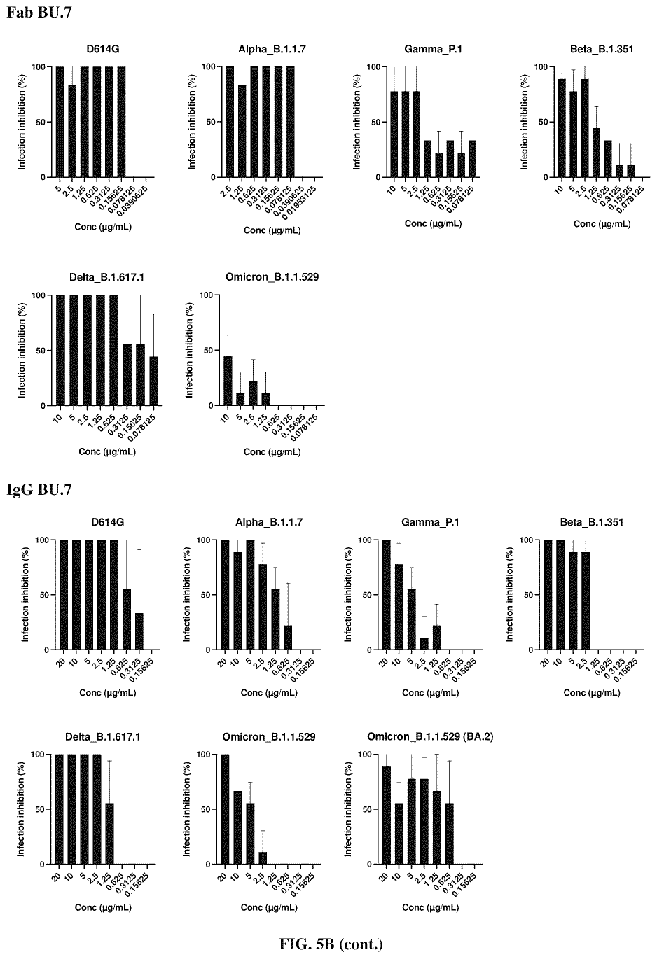

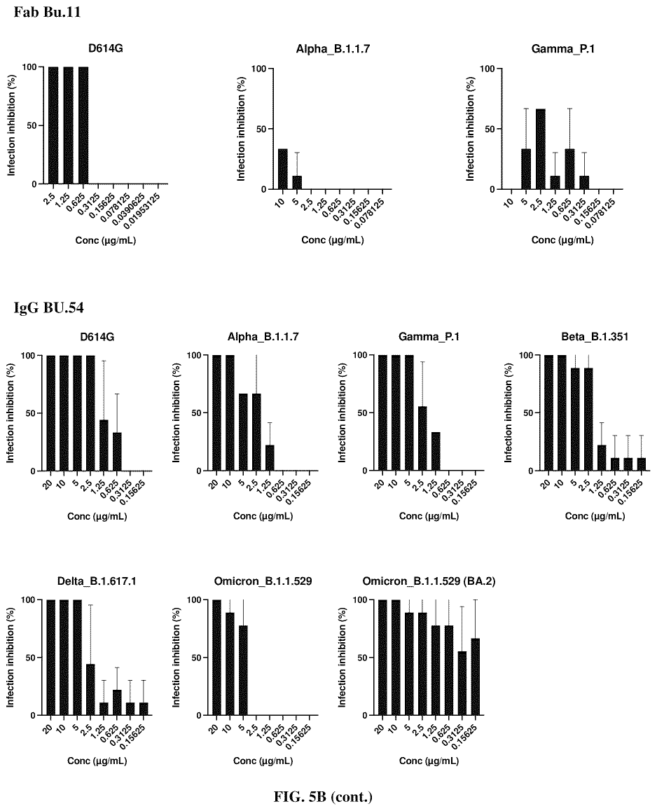

FIG. 5 shows neutralizing activities of clone Fab #5, both as Fab and IgG1 format against the tested VOCs (D614G, B.1.1.7, P.1 and B.1.135) (panel A) and of clones Fab BU.2, BU.7, BU.11, BU.54 and BS.70 both as Fab and IgG1 format (panel B) against the tested VOCs (D614G, Alpha_B.1.1.7, Beta_B.1.135, Delta B.1.617.1, Gamma_P.1, Omicron B.1.1.529 (BA.1), Omicron B.1.1.529 (BA.2)).

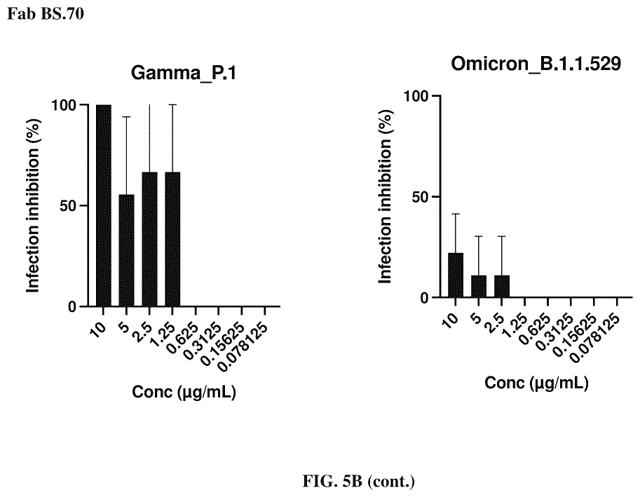

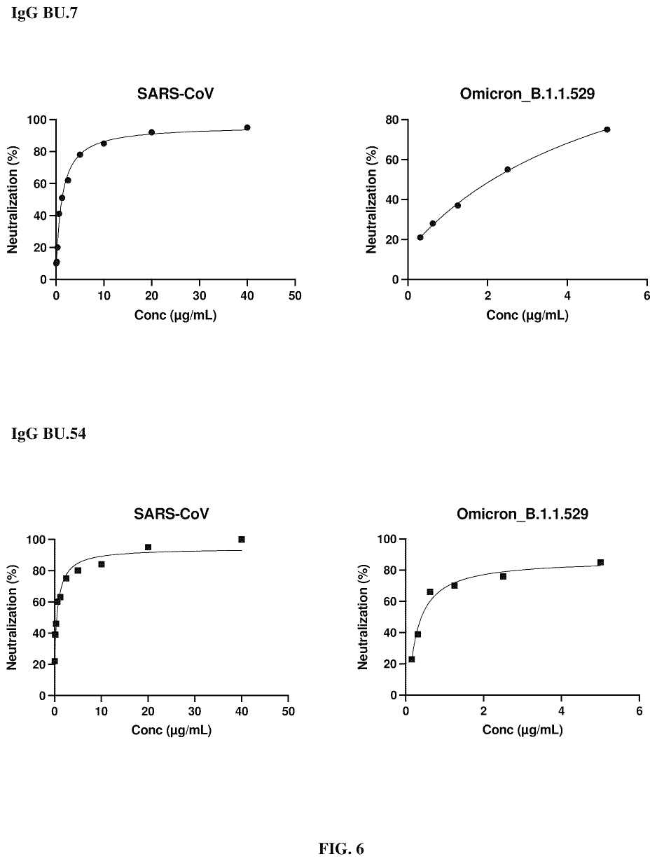

FIG. 6 shows neutralizing activity using pseudovirus of clones Fab BU.7 and BU.54 against SARS-CoV-1 and Omicron VOC of SARS-CoV-2.

For the purpose of better illustrating the invention, the following examples are now provided, which should be considered illustrative and non-limiting examples thereof. The neutralizing nAbs of the invention have been identified on the basis of the binding affinity to SARS-CoV1 and SARS-CoV-2 spike or specific domains and characterized in terms of neutralizing activity and epitope specificity as described in the following examples. These aspects are fundamental to select the most promising nAbs as those endowed with the highest potency and/or recognizing conserved spike epitopes, the main parameters determining a potential cross reactive treatment effective against all the VOIs and VOIc of SARS-Cov-2 known sofar or emerging in the future.

### Example 1: Generation of New Human Antibody Phage Display Libraries and Selection of Antibodies Able to Bind with High Affinity the Spike Protein of SARS-CoV-2 Through Bio-Panning Procedures

### Materials and Methods

### Phage Display Technique

The technique focuses on the construction of a library of peptides or antibody variants, which are then selected for their affinity to the target of interest since they are fused to a phage-coat protein (see for reference FIG. 1).

All surface proteins of bacteriophages can be engineered for display, but the most used are pVIII and pIII from M13 filamentous phages. Each virion contains about 2700 copies of the former protein, representing almost 87% of its mass, and they are half-exposed to the environment, thus allowing an efficient display of only short-sequence peptides due to virion architecture. On the other hand, pIII can be used for larger peptides, such as the “functional portion” of antibodies, resulting only in a slight loss of infectivity in a few cases. Each library is composed by phagemid vectors containing only the sequence of a phage-coat protein fused to the peptide of interest; therefore, a helper phage with a reduced packaging efficiency is needed to obtain a population of phages both infectious and composed by modified coating proteins. The bio-panning procedure is then performed, and phages are selected for their ability to bind the antigen of interest. Many factors must be considered: library variability, target conformation, affinity, and avidity of the molecules exposed on phages. As mentioned, phage display has been widely used to find novel therapeutics against pathogens, particularly mAbs.

This has been possible through two different bio-panning strategies: using specific molecular targets, such as membrane receptors (S protein), or using whole viruses, or infected cells. However, surface antigens often present motifs able to elicit non-neutralizing mAbs and elude host immune response, so the screening procedure of the bio-panning outcome must be done properly to identify only the few effective molecules able to selectively target the antigen of interest.

### Generation of New Human Antibody Phage Display Libraries

Peripheral (for Fab #5) and bone marrow (for Fabs BU.2, BU.7, BU.11, BU.54) blood samples was collected from subjects previously infected by SARS-CoV-2 and subsequently vaccinated with COVID-19 vaccine (2 doses of Comirnaty, Pfizer) and used for the collection of PBMC and B-cell precursors, respectively. The extraction of PBMCs was carried out following the Histopaque®-1077 (SIGMA-ALDRICH) protocol. At the end of the procedure 10⁶ cells were lysed using Trizol Reagent (SIGMA-ALDRICH) to allow the subsequent extraction of cellular RNA using RNeasy Mini Kit (QIAGEN). Antibody phagemidic IgG libraries were generated through B-cell-mRNA reverse transcription to cDNA using SuperScript™ IV Reverse Transcriptase (ThermoFisher Scientific). Heavy and light chains were amplified from the obtained cDNA by using the PFU Native Polymerase enzyme (Agilent) and the primer pairs described in the following Table.

TABLE 1
Primers used for the amplification of
heavy and light chains

| | Primer Forward | Primer Reverse |
|---|---|---|
| Heavy | | |
| chain | | |
| 1 | VH135: AGGTGCAG | IgG1: GCATGTACT |
| | CTGCTCGAGTCTGG | AGTTTTGTCACAAGA |
| | (SEQ ID NO: 17) | TTTGG |
| | | (SEQ ID NO: 18) |
| 2 | VH2: CAGATCACCT | IgG1 |
| | TGCTCGAGTCTGG | |
| | (SEQ ID NO: 19) | |
| 3 | VH4: CAGGTGCAGC | |
| | TGCTCGAGTCGGG | |
| | (SEQ ID NO: 84) | |
| 4 | VH4b: CAGGTGCAG | |
| | CTACTCGAGTGGGG | |
| | (SEQ ID NO: 85) | |
| 5 | VH4gs: CAGGTGCA | |
| | GCTACTCGAGTGGGG | |
| | C | |
| | (SEQ ID NO: 86) | |
| 6 | VH6: CAGGTACAGC | |
| | TGCTCGAGTCAGG | |
| | (SEQ ID NO: 87) | |
| Light | | |
| chain | | |
| 1 | VK1: GAGCCGCACG | CK1d: GCGCCGTCT |
| | AGCCCGAGCTCCAGA | AGAATTAACACTCTC |
| | TGACCCAGTCTCC | CCCTGTTGAAGCTCT |
| | (SEQ ID NO: 88) | TTGTGACGGGCGAAC |
| | | TCAG |
| | | (SEQ ID NO: 89) |
| 2 | VK3: GAGCCGCACG | CK1d |
| | AGCCCGAGCTCGTGA | |
| | TGACACAGTCTCC | |
| | (SEQ ID NO: 90) | |
| 3 | VK2/4: GAGCCGCA | |
| | CGAGCCCGAGCTCGT | |
| | GATGACCCAGTCTCC | |
| | (SEQ ID NO: 91) | |
| 4 | VK5: GAGCCGCACG | |
| | AGCCCGAGCTCACAC | |
| | TCACGCAGTCTCC | |
| | (SEQ ID NO: 92) | |

Then, both heavy and light variable antibody chains were cloned into the phagemidic vector pComb3XSS (PVT10572, DBA).

Phagemidic libraries were then converted into phage libraries by transforming electrocompetent Gram-negative cells expressing sexual pilus (XL1-Blue, Agilent) and superinfecting them with a helper-phage (M13K07, NEB).

The new libraries were screened by using different Bio-panning procedures with the aim of maximizing the selection of cross-reactive antibodies (antibodies recognizing more SARS-CoV-2 clinical isolates) in their Fab format.

Recombinant spike(S) proteins of different SARS-CoV-2 variants were used: D614G (University of Pavia) for Fab #5, and B.1.1.7 (40589-V08B6, Sino Biological), P.1 (40589-V08B10, Sino Biological) or B.1.135 (40589-V08B7, Sino Biological) for Fab BU.2, BU.7, BU.11, BU.54.

Five rounds of Bio-panning of the phage-display antibody libraries were carried out on recombinant Spike proteins expressed in their full-length format. Cross-selection strategies based on the sequential use of the different antigens were performed.

Bacteriophages carrying on their capsid monoclonal antibodies (expressed as Fab fragments) selected through bio-panning were used to obtain the DNA codifying the antibody expressed on their surface.

DNAs codifying for the variable antibody regions present on the phage surface was sequenced and used for protein purification using E. coli expression system (XL1-Blue, Agilent) and immune-affinity chromatography (HiTrap® Protein L, Merck). IgG1 format of Fab #5 (IgG #5) was obtained from Genscript through High Throughput Antibody Production Service (GenScript).

### Results

Five different clones were identified: Fab #5 and Fab BU.2, BU.7, BU.11, BU.54. The amino acidic sequences of the heavy and light chain variable regions are the following:

1. Fab #5
Hc:
(SEQ ID NO: 60)
LESGGGLVKPGGSLRLSCAASGFNFNTYTMNWVRQAPGKGLEWVSSISSSS
SYIDNADSVKGRFTIYRDNAKKSLYLRMIGLRVEDSGVYYCTRVNPQAKGS
DWLDPPINQYYGMDVWGQGTTVTVSS
Lc:
(SEQ ID NO: 61)
ELTLTQSPGTLSLSPGERATLSCRASQSVSSSYLAWYQQKPGQAPRLLIYGA
SSRATGIPDRFSGSGSGTDFTLTISRLEPEDFAVYYCQQYGSSPWTFGQGTK
VEIK
2. Fab BU.2
Hc:
(SEQ ID NO: 62)
LESGGGLVKPGGSLRLSCAASGFNFNTYTMNWVRQAPGKGLEWVSSISSSS
SYIDNADSVKGRFTIYRDNAKKSLYLRMIGLRVEDSGVYYCTRVNPQAKGS
DWLDPPINQYYGMDVWGQRDHGHRSP
Lc:
(SEQ ID NO: 63)
ELTLTQSPATLSLSPGDRATLSCRASQSVSSSYLAWYQHKPGQPPRLLIYGA
SSRAAGIPDRFSGSGSGTDFTLTISRLEPEDFAVYYCQQYVTFGPGTKVDIK
3. Fab BU.7
Hc:
(SEQ ID NO: 64)
LEWGPGLVKPSETLSLTCTVSGGSISSINYYWVWIRQPPGKGLEWIGNINYS
GTTNYNPSLKSRVTISVDTSKNQFSLKLSSVTAADTAVYYCARQTYYYDRR
GYYRPEPIEHWGQGTLVTVSS
Lc:
(SEQ ID NO: 65)
ELVMTQSPSFLSASVGDRVTITCRASQDVRSFLHWYQQRPGKAPKLLIYAA
SMVSSEVPSRFSGSGSETDFTLTIDGLQPEDVATYFCQQTYDTPLTFGGGTA
VDIK
4. Fab BU.11
Hc:
(SEQ ID NO: 66)
LESGPGLLKPSQSLSLTCAISGDSVSRRSVAWNWIRQSPSRGLEWLGRTYYR
SKWFSEYGVSVRGRITISPDTTKNQFSLQLNSVTPEDTAVYYCRKSKGRQQ
LAESTSSVWTWGQGTTVIVYS
Lc:
(SEQ ID NO: 67)
ELVMTQSPSSLSAFVGDRVTLTCRASQGIRNDLNWYQQKPGQPPKLLIYAA
SALQSGVPSRFSGSGFGTDFTLTISSLQPEDIATYYCLQDYNFPRTFGQGTKV
EIK
5. Fab BU.54
Hc:
(SEQ ID NO: 68)
LEWGPGLVKASQTLSLTCTVSGGSISSRNFYWSWIRQPGGKGLEWIGRIYTS
GSTNYNPSLKSRVTISLDTSKSQFSLKLSSVTAADTAVYYCARGTFYYDRSG
NGRLDPLDYWGQGTLVTVSS
Lc:
(SEQ ID NO: 69)
ELVMTQSPSSLSASVGDRVTITCQASQDISNYLNWYQQKPGKAPKLLIYDA
SNLETGVPSRFSGSGFGTHFTLTISSLQPEDFATYYCQQHDNLVTFGGGTKV
EIK
6. Fab BS.70
Hc:
(SEQ ID NO: 70)
LESGGGVVQPGTSLRLSCAASGFTFNNFGMHWVRQAPGKGLEWVAMISYE
GSKDFYADSVKGRFTISKDHARNTVYLQMNSLRAEDTAEYYCAKDKAIFM
ISAGRTLDFWGQGTLVTVSS
Lc:
(SEQ ID NO: 71)
ELVMTQSPSSLSASVGDRVTITCRASQNIGIYLNWYQQKPGKAPKLLIYAAS
SLQNGVPSRFSGSGSGTDFTLTISTLQPEDFATYWCQQGYSTPLTFGGGTKV
EIR
The nucleotide sequences encoding the same are the following:
1. Fab #5
Hc:
(SEQ ID NO: 72)
ctcgagtctgggggaggcctggtcaagcctggggggtccctgagactctcctgtgcagcctctggattcaacttcaatac
ctataccatgaactgggtccgccaggctccagggaaggggctggagtgggtctcatccattagtagtagtagtagttaca
tagacaacgcagactcagtgaagggccgattcaccatctacagagacaacgccaagaagtcactgtatctgcgaatgat
cggcctgagagtcgaggactccggtgtgtattactgtacgcgagtgaacccccaggccaaagggtcggactggttgga
tccccccatcaaccaatactacggtatggacgtctggggccaagggaccacggtcaccgtctcctca
Lc:
(SEQ ID NO: 73)
gagctcacactcacgcagtctccaggcaccctgtctttgtctccaggggaaagagccaccctctcctgcagggccagtc
agagtgttagcagcagctatttagcctggtaccagcagaaacctggccaggctcccaggctcctcatctatggtgcatcc
agcagggccactggcatcccagacaggttcagtggcagtgggtctgggacagacttcactctcaccatcagcagactg
gagcctgaagattttgcagtgtattactgtcagcagtatggtagctcaccgtggacgttcggccaagggaccaaggttga
aatcaaa
2. Fab BU.2
Hc:
(SEQ ID NO: 74)
ctcgagtctgggggaggcctggtcaagcctggggggtccctgagactctcctgtgcagcctctggattcaacttcaatac
ctataccatgaactgggtccgccaggctccagggaaggggctggagtgggtctcatccattagtagtagtagtagttaca
tagacaacgcagactcagtgaagggccgattcaccatctacagagacaacgccaagaagtcactgtatctgcgaatgat
cggcctgagagtcgaggactccggtgtgtattactgtacgcgagtgaacccccaggccaaagggtcggactggttgga
tccccccatcaaccaatactacggtatggacgtctggggccaaagggaccacggtcaccggtctcct
Lc:
(SEQ ID NO: 75)
gagctcacactcacgcagtctccagccaccctgtctttgtctccaggggatagagccaccctctcctgcagggccagtc
agagtgttagcagcagttacttagcctggtaccagcacaaacctggccagccccccaggctcctcatctatggtgcatcc
agcagggccgccggcatcccagacaggttcagtggcagtgggtctgggacagacttcactctcaccatcagcagact
ggagcctgaagattttgcagtgtattactgtcagcagtacgtcactttcggccctgggaccaaagtggatatcaaa
3. Fab BU.7
Hc:
(SEQ ID NO: 76)
ctcgagtggggcccaggactggtgaagccttcggagaccctgtccctcacctgcactgtctctggtggctccatcagca
gcataaattactactgggtctggattcgccagcccccagggaaggggctggagtggattgggaatatcaattacagtgg
gaccaccaactacaacccgtccctcaagagtcgagtcaccatatccgtggacacgtccaagaaccagttctccctgaag
ctgagctctgtgaccgccgcagacacggctgtctattactgtgcgagacaaacgtattactatgataggcgtggttattac
cgcccggagcccattgagcactggggccagggaaccctggtcaccgtctcctca
Lc:
(SEQ ID NO: 77)
gagctcgtgatgacacagtctccatccttcctgtctgcatctgtaggagacagagtcaccatcacttgccgggcaagtca
agatgttagaagttttttacattggtatcaacaaagaccagggaaagcccctaagttgttgatctatgctgcatccatggtgt
cgagtgaggtcccgtcaaggttcagtggcagtggatctgagacagatttcactctcaccatcgacggtctgcaacctgaa
gatgttgcaacttacttctgtcaacagacttacgacacgccgctcaccttcggcggcgggaccgcggttgacatcaaa
4. Fab BU.11
Hc:
(SEQ ID NO: 78)
ctcgagtcaggtccaggactgctgaagccctcgcagagcctctcactcacctgtgccatctccggagacagtgtctcta
ggagaagtgttgcttggaactggatcagacagtccccatcgagaggccttgagtggctgggaaggacatactacaggt
ccaagtggttttctgagtatggcgtatctgtgagaggtcgaataaccatcagtccagacacaaccaagaaccagttctcc
ctgcagctgaactccgtgactcccgaggacacggctgtctattactgtcgaaagtcgaaagggaggcagcagttggca
gagtctacgagttcggtatggacgtggggccaagggaccacggtcatcgtctactca
Lc:
(SEQ ID NO: 79)
gagctcgtgatgacacagtctccatcctccctgtctgcatttgtgggagatagagtcaccctcacttgccgggcaagtca
gggcattagaaatgatttaaactggtatcagcagaaaccagggcaaccccctaaactcctgatctatgctgcatccgcttt
acaaagtggcgtcccatcaaggttcagcggcagtgggtttggcacagatttcactctcaccatcagcagcctgcagcct
gaagatattgcaacttattactgtctacaagattacaatttccctcggacgttcggccaagggaccaaggtggaaatcaaa
5. Fab BU.54
Hc:
(SEQ ID NO: 80)
ctcgagtggggcccaggactggtgaaggcttcacagaccctgtccctcacctgcactgtctctggtggctccatcagca
gtaggaatttctattggagttggatccggcagcccggcgggaagggactggagtggattgggcgtatctatacaagtgg
gagcaccaattacaatccctccctcaagagtcgagtcactatatcattagacacgtccaagagtcagttctccctgaagct
gagctctgtgaccgccgcagacacggccgtgtattattgtgcgagagggacgttttattatgataggagtggtaatggtc
gattagatccgcttgactactggggccagggaaccctggtcaccgtctcctca
Lc:
(SEQ ID NO: 81)
gagctcgtgatgacacagtctccatcctccctgtctgcatctgtaggagacagagtcaccatcacttgccaggcgagtca
ggacattagcaactatttaaattggtatcagcagaaaccagggaaagcccctaagctcctgatctacgatgcatccaattt
ggaaacaggggtcccatcaaggttcagtggaagtggatttgggacacattttacgttaaccatcagcagcctgcagcct
gaagattttgcaacatattactgtcaacagcatgataatctcgtcactttcggcggagggaccaaggtggagatcaaa
6. Fab BS.70
Hc:
(SEQ ID NO: 82)
ctcgagtctgggggaggcgtggtccagcctgggacgtccctgagactctcctgtgcggcctctggattcaccttcaataa
ttttgggatgcactgggtccgccaggctccaggcaagggtctggagtgggtggcaatgatctcatatgaaggaagtaag
gatttctatgcagactccgtgaagggccgattcaccatctccaaagaccatgccaggaatacggtctatctgcaaatgaa
cagcctgagagctgaggacacggcagaatattactgtgcgaaagataaggctatatttatgatttctgccggacggacttt
ggacttctggggccagggaaccctggtcaccgtctcctcag
Lc:
(SEQ ID NO: 83)
gagctcgtgatgacacagtctccatcctccctgtctgcatctgtaggagacagagtcaccattacttgccgggcaagtca
gaacattggcatctatttgaattggtatcagcagaaacctgggaaagccccaaagctcctgatctatgctgcatccagtttg
caaaatggggtcccatcgaggttcagtggcagtggatctgggacagacttcactctcaccatcagcactctgcaacctg
aagattttgcaacttactggtgtcaacagggttacagtaccccactcactttcggcggagggaccaaggtagagatcaga
c

FIG. 2 shows the whole amino acid and nucleotide sequence of the heavy chain variable region and of the light chain variable region for each clone, specifying both the framework regions FR1-FR4 and the complementary determining regions CDR1-CDR3.

### Example 2: Study on the Binding Activity of the Neutralizing mAbs of the Invention

### Binding Evaluation of the Fabs

### a) Enzyme Linked Immunosorbent Assay (ELISA)

The binding efficiency of the Fabs was tested using ELISA assay.

100 ng of the recombinant Spike protein D614G (University of Pavia), B.1.1.7 (40589-V08B6, Sino Biological), P.1 (40589-V08B10, Sino Biological), or B.1.135 (40589-V08B7, Sino Biological) were coated on ELISA plates (CLS3690-100EA, Merck) and incubated overnight at 4° C. 1% bovine serum albumin (BSA; Sigma-Aldrich) solution in PBS was added as a nonspecific reactivity control. The following day, the plate was blocked with a 1% BSA solution in PBS to prevent nonspecific binding to wells, and different dilutions of Fabs were incubated with antigens for 1 h at 37° C. After washing with 0.1% Tween 20 (Sigma-Aldrich) in PBS solution, Fabs were detected with goat anti-human IgG (Fab specific)-peroxidase antibody (A0293; Sigma-Aldrich), with His-tagged proteins with anti-His6-peroxidase antibody (11
2416001; Sigma-Aldrich) as a coating control and using the Pierce TMB substrate kit (ThermoFisher Scientific). The OD450 was measured using Multiskan GO (ThermoFisher Scientific).

### Results

Fab #5 binds the S protein of Alpha, Gamma, and Omicron
IgG #5 binds the S protein of D614G, Alpha, Beta, Gamma, Delta, Omicron, and SARS-CoV-1
Fab BU.2 binds the S protein of Alpha
Fab BU.7 binds the S protein of Alpha, SARS-CoV-1, and less Gamma, Omicron
IgG BU.7 little binds the S protein of Omicron
Fab BU.11 binds the S protein of Alpha
IgG BU.54 binds the S protein of Alpha, Delta, Omicron, and SARS-CoV-1
Fab BS.70 binds the S protein of Alpha, Delta, Omicron, and SARS-CoV-1

Results are depicted in FIG. 3. Data showed that Fab #5 can recognize the B.1.1.7 recombinant protein even when used at low concentrations, but also P.1 spike when used at higher concentrations. IgG #5 binds well the recombinant spike of all tested SARS-CoV-2 variants (D614G, B.1.1.7, B.1.351, P.1, B.1.1.529, B.1.617.1). The BU.7 binds P.1 protein, and even better B.1.1.7 spike protein. Its IgG format shows a weak binding activity also on B.1.617.1 and B.1.1.529 proteins. Moreover, B.1.1.7 spike is well recognized also by BU.11 and BU.2 Fabs, and IgG BU.54 recognizes well B.11.7, B.1.617.1, P.1, B.1.1.529 and SARS-CoV-1 recombinat proteins. Fab BS.70 binds even at low concentrations B.1.1.7, B.1.617.1, B.1.1.529 proteins. Last, SARS-CoV-1 spike protein is very well recognized by IgG #5, BU.7 in both Fab and IgG formats, IgG BU.54, and Fab BS.70.

### b) Immunofluorescence Analysis

### Virus and Cells

Vero E6 (Vero C1008, clone E6-CRL-1586; ATCC) cells were cultured in Dulbecco's modified Eagle's medium (DMEM) supplemented with non-essential amino acids (NEAA), penicillin/streptomycin (P/S), Hepes buffer, and 10% (v/v) fetal bovine serum (FBS). Four clinical isolates of SARS-CoV-2 were obtained and propagated in Vero E6 cells: D614G (hCoV-19/Italy/UniSR1/2020; GISAID Accession ID: EPI_ISL_413489), B.1.1.7 (Alpha) (19/Italy/LOM-UniSR7/2021; GSAID Accession ID: EPI_ISL_1924880), C.36_3 (hCoV-19/Italy/LOM-UnINSU/2021, GISAID Accession ID: EPI_ISL_1509923), B.1.351 (Beta) (hCoV-19/Italy/LOM-UniSR6/2021, GISAID Accession ID: EPI_ISL_1599180), P.1 (Gamma) (hCoV-19/Italy/LOM-UniSR8/2021, GISAID Accession ID: EPI_ISL_1925323) Delta (hCoV-19/Italy/LOM-UniSR12/2021, GSAID accession ID: EPI_ISL_4198505), Omicron BA.1 (hCoV-19/Italy/LOM-UniSR14/2021, GSAID accession ID: EPI_ISL_12188061), Omicron BA.2 (hCoV-19/Italy/LOM-UniSR15/2022. GISAID accession ID: EPI_ISL_13285445), Omicron BA.4 (hCoV-19/Italy/LOM-UniSR17/2022. GISAID accession ID: EPI_ISL_13878328) and Omicron BA.5.2 (hCoV-19/Italy/LOM-UniSR16/2022. GISAID accession ID: EPI_ISL_13878326).

### Virus Titration

Virus stocks were titrated using both Plaque Reduction Assay (PRA, PFU/ml) and Endpoint Dilutions Assay (EDA, TCID50/ml). In PRA, confluent monolayers of Vero E6 cells were infected with eight 10-fold dilutions of virus stock. After 1 h of adsorption at 37° C., the cell-free virus was removed. Cells were then incubated for 48 h in DMEM containing 2% FBS and 0.5% agarose. Cells were fixed and stained, and viral plaques were counted. In EDA, Vero E6 cells were seeded into 96 wells plates and infected at 95% of confluency with base 10 dilutions of virus stock. After 1 h of adsorption at 37° C., the cell-free virus was removed, cells were washed with PBS 1×, and complete medium was added to cells. After 48 h, cells were observed to evaluate the presence of a cytopathic effect (CPE). TCID50/ml of viral stocks were then determined by applying the Reed-Muench formula.

### Immunofluorescence Assay

Vero E6 cells were seeded into 96 wells plates 24 h before the experiment performed at 95% cell confluency for each well, and then infected with SARS-CoV-2 at 0.01 multiplicity of infection (MOI) for 1 h at 37° C. Then, the cells were washed with PBS 1× to remove cell-free virus particles and virus-containing mixtures and controls were replaced with complete DMEM supplemented with 2% FBS. The plates were incubated at 37° C. in the presence of CO2 for 72 h. Then were washed with PBS 1× and fixed with MeOH: Ac (1:1) for 15 minutes, and stored at −20° C. After rinsing the cells with two PBS 1× washes, the selected Fabs (1:200 dilution) were applied to cells as primary antibody. Anti-Spike Antibody (0150-R007, Sino Biological) was added as control. After a PBS 1× wash, Fabs were detected with goat anti-rabbit IgG (H+L) cross-adsorbed secondary antibody, Alexa Fluor 488 (A-11008, Thermo Scientific). Hoechst 33258 (Sigma-Aldrich) was used for nuclear staining.

### Results

All the selected Fabs detected the infected cells, with slight differences between the tested VOCs (FIG. 4).
Fab #5 binds cells infected with D614G, Alpha, Beta, Gamma, Delta, Omicron (BA.1), and Omicron (BA.2)
IgG #5 binds cells infected with D614G, Alpha, Beta, Gamma, Delta, Omicron (BA.1), Omicron (BA.2), Omicron (BA.5), and less Omicron (BA.4)
Fab BU.2 binds cells infected with D614G, Alpha, Gamma, Delta, Omicron (BA.2), less Beta, and no Omicron (BA.1)
Fab BU.7 binds cells infected with D614G, Alpha, Beta, Gamma, Delta, Omicron (BA.1), and Omicron (BA.2)
IgG BU.7 binds cells infected with D614G, Alpha, Beta, Gamma, Delta, Omicron (BA.1), Omicron (BA.2), Omicron (BA.5), and Omicron (BA.4).
Fab BU.11 binds cells infected with D614G, Alpha, Gamma, Delta, Beta, less Omicron (BA.2), and no Omicron (BA.1)
IgG BU.54 binds cells infected with D614G, Alpha, Beta, Gamma, Delta, Omicron (BA.1), Omicron (BA.2), Omicron (BA.5), and less Omicron (BA.4)
Fab BS.70 binds cells infected with D614G, Alpha, Beta, Gamma, Delta, Omicron (BA.1), and Omicron (BA.2), (BA.4) and less (BA.5)

B.1.1.7 variant is recognized better by Fab #5, BU.2, BU.11 and IgG BU.54, and less well by BS.70 and BU.7 Fabs. However, the IgG format of BU.7 shows an improved binding activity against this variant. B.1.135 variant is recognized well only by clone #5 in both Fab and IgG formats, IgG BU.7, IgG BU.54, and Fab BS.70, while only a slight green signal is observed using the other Fabs. The binding to the P.1 variant is well observed with all the tested Fabs, but the Fab #5 signal had the least intensity. Delta variant is well recognized by all Fabs and IgGs tested, while Omicron (BA.1) is not recognized only by BU.2 and BU.11 Fabs. Last, Omicron (BA.2) is recognized, albeit with a less intense signal than BA.1, by all but Fab BU.11. Fab BS.70 is the clone showing the strongest binding signals against all tested variants, followed by IgG #5, IgG BU.7, IgG BU.54. Both Omicron BA.4 and BA.5 are well recognized by IgGBU.7 on infected cells. IgG #5 and IgG BU.54 strongly recognize Omicron BA.5 sublineage and recognize with lower binding signal the BA.4.

### Example 3: Neutralizing Activity Evaluation

### Microneutralization Experiments

Vero E6 cells were seeded into 96 wells plates 24 h before the experiment performed at 95% cell confluency for each well. Two-fold serial dilutions of the selected clones were incubated with SARS-CoV-2 at 0.01 MOI for 1 h at 37° C. Virus-serum mixtures and positive infection control were applied to Vero E6 monolayers after washing cells with PBS 1×, and virus adsorption was carried out at 37° C. for 1 h. Then, the cells were washed with PBS 1× to remove cell-free virus particles and virus-containing mixtures and controls were replaced with complete DMEM supplemented with 2% FBS. The plates were incubated at 37° C. in the presence of CO₂ for 72 h. The experiments were performed in triplicate. Neutralization activity was evaluated by comparing cytopathic effect (CPE) presence detected in the presence of virus-serum mixtures to positive infection control.

### Results

The clone #5 showed an important neutralizing activity against D614G variant both as Fab format and IgG1. However, IgG1 showed enhanced activity against the other tested VOCs compared to the Fab fragment, except for B.1.135 variant (FIG. 5A). All tested BU clones can inhibit efficiently D614G, but only BU.7 retains its activity against all the tested VOCs. Fab BU.2 can neutralize better B.1.1.7 variant than the other two, while BU.11 showed a significant activity against P.1 (FIG. 5B).

In detail:
Fab #5 neutralizes D614G and to a lesser extent Alpha and Gamma
IgG #5 neutralizes D614G, Alpha, Gamma, and less Beta
Fab BU.2 neutralizes D614G, Alpha, Delta, and less Gamma and Beta
Fab BU.7 neutralizes D614G, Alpha, Gamma, Beta, Delta, and less Omicron
IgG BU.7 neutralizes D614G, Alpha, Gamma, Beta, Delta, Omicron, and Omicron (BA.2)
Fab BU.11 neutralizes D614G, and less Alpha and Gamma
IgG BU.54 neutralizes D614G, Alpha, Gamma, Beta, Delta, Omicron, and Omicron (BA.2)
Fab BS.70 neutralizes Gamma, and less Omicron

The following Tables 2-3-4 summarize the neutralizing activity expressed as IC₅₀ μg/ml shown by the selected clones Fab #5, IgG #5, Fab BU.2, Fab BU.7, Fab BU.11, Fab BS.70, IgG BU.7, IgG BU.54 against different VOCs of SARS-CoV-2.

TABLE 2

| | Fab#5 (μg/ml) | IgG#5 (μg/ml) |
|---|---|---|
| D614G | 0.904 | 8.153 |
| B.1.1.7 | 1.422 | 2.176 |
| P.1 | 4.459 | 6.04 |

TABLE 3

| | Fab | Fab | Fab | Fab |
|---|---|---|---|---|
| | BU.2 (μg/ml) | BU.7 (μg/ml) | BU.11 (μg/ml) | BS.70 (μg/ml) |
| D614G | 0.002935 | 0.0985 | 0.4075 | >10 |
| B.1.1.7 | 5.2 | 0.67425 | 5.3525 | >10 |
| P.1 | 0.31205 | 0.4426 | 0.3182 | 1.047 |
| B.1.351 | >10 | 0.955 | >10 | >10 |

TABLE 4

| | IgG #5 (μg/ml) | IgG BU.7 (μg/ml) | IgG BU.54 (μg/ml) |
|---|---|---|---|
| D614G | 8.153 | 0.024 | 0.925 |
| B.1.1.7 | 2.176 | 0.856 | 2.156 |
| B.1.351 | 2.394 | 2.371 | 1.708 |
| P.1 | 6.04 | 5.816 | 1.998 |
| B.1.617.2 | >20 | 1.241 | 2.597 |
| B.1.1.529 | >20 | 5.828 | 4.313 |

### Example 4: Neutralization Assays Using Pseudovirus

### Methods

SARS-CoV-2-pseudotyped particles were produced and titrated.

Briefly, HEK293 T were co-transfected with the S plasmids from either SARS-CoV-1 or SARS-CoV-2 Omicron, HIV gag-pol and the Luc-reporter plasmid.

Supernatant has been harvested 72 h after the transfection, clarified, and filtered. Viral titers were evaluated on HEK293T ACE2-TMPRSS2 cells with two-fold serial dilutions pseudoviral stock. After 48 h (37° C. in the presence of 5% CO₂) luciferase expression was quantified. Titers were calculated as RLU/ml.

### Surrogate Neutralization Assays

Neutralization assays were performed by incubating pseudoviruses (106 RLU) with endpoint twofold serial dilutions of monoclonal antibodies (37° C. in the presence of 5% CO₂) for 1 h before adding the neutralization mix of 104 HEK 293T-ACE2-TMPRSS2 cells per well. 72 h post-infection (37° C.), luciferase activity was quantified.

### Results

The neutralization assay according to this alternative strategy show that the monoclonal antibodies of the invention IgG BU.7 and IgG BU.54 neutralize SARS-CoV-1 and Omicron variant of SARS CoV-2. FIG. 6 shows the results of the neutralization assays using pseudovirus.

The following Table 5 summarize the neutralizing activity expressed as IC₅₀ μg/ml shown by the selected clones IgG BU.7 and IgG BU.54 against SARS-CoV-1 and Omicron variant of SARS CoV-2.

TABLE 5

| | IgG BU.7 (μg/ml) | IgG BU.54 (μg/ml) |
|---|---|---|
| SARS-COV-1 | 1.3 | 0.4 |
| B.1.1.529 | 1.8 | 0.5 |

### REFERENCES

1. Lanying Du et al. (2021) Neutralizing antibodies for the prevention and treatment of COVID-19. Cellular and Molecular Immunology, 18, pp. 2293-2306. doi:10.1038/s41423-021-00752-2
2. An EUA for Bamlanivimab—A Monoclonal Antibody for COVID-19. JAMA. 2021; 325(9):880-881. doi:10.1001/jama.2020.24415
3. An EUA for bamlanivimab and etesevimab for COVID-19. Med Lett Drugs Ther, 2021 Apr. 5; 63(1621):49-50. pmid:33830966
4. An EUA for casirivimab and imdevimab for COVID-19. Med Lett Drugs Ther. 2020 Dec. 28; 62(1614):201-2. pmid:33451174
5. Dong J, Zost S J, Greaney A J, Starr T N, Dingens A S, Chen E C, et al. Genetic and structural basis for recognition of SARS-CoV-2 spike protein by a two-antibody cocktail. bioRxiv; doi:10.1101/2021.01.27.428529
6. Cathcart A L, Havenar-Daughton C, Lemp F A, Ma D, Schmid M A, Agostini M L, et al. The dual function monoclonal antibodies VIR-7831 and VIR-7832 demonstrate potent in vitro and in vivo activity against SARS-CoV-2. bioRxiv; doi: 10.1101/2021.03.09.434607
7. Kim C, Ryu D-K, Lee J, Kim Y-I, Seo J-M, Kim Y-G, et al. A therapeutic neutralizing antibody targeting receptor binding domain of SARS-CoV-2 spike protein. Nat Commun. 2021 Jan. 12; 12(1):288. doi:10.1038/s41467-020-20602-5
8. Alsoussi W B, Turner J S, Case J B, Zhao H, Schmitz A J, Zhou J Q, et al. A Potently Neutralizing Antibody Protects Mice against SARS-CoV-2 Infection. J Immunol. 2020 Aug. 15; 205(4):915-22. doi:10.4049/jimmunol.2000583
9. Wang C, Li W, Drabek D, Okba N M A, van Haperen R, Osterhaus A D M E, et al. A human monoclonal antibody blocking SARS-CoV-2 infection. Nat Commun. 2020 May 4; 11(1):2251. doi:10.1038/s41467-020-16256-y
10. Tzou P L, Tao K, Nouhin J, Rhee S-Y, Hu B D, Pai S, et al. Coronavirus Antiviral Research Database (CoV-RDB): An Online Database Designed to Facilitate Comparisons between Candidate Anti-Coronavirus Compounds. Viruses. 2020 September; 12(9):1006. doi:10.3390/v12091006
11. Kumar S. et al. (2021) Current status of therapeutic monoclonal antibodies against SARS-CoV-2. PLoS Pathog 17(9):e1009885. doi:10.1371/journal.ppat.1009885
12. Frampton D, Rampling T, Cross A, Bailey H, Heaney J, Byott M, et al. Genomic characteristics and clinical effect of the emergent SARS-CoV-2 B.1.1.7 lineage in London, UK: a whole-genome sequencing and hospital-based cohort study. Lancet Infect Dis. 2021 September; 21(9):1246-1256. doi:10.1016/S1473-3099(21)00170-5.
13. Tegally H, Wilkinson E, Giovanetti M, Iranzadeh A, Fonseca V, Giandhari J, et al. Emergence and rapid spread of a new severe acute respiratory syndrome-related coronavirus 2 (SARS-CoV-2) lineage with multiple spike mutations in South Africa. medRxiv; doi: 10.1101/2020.12.21.20248640
14. Voloch C M, da Silva F R, de Almeida L G P, Cardoso C C, Brustolini O J, Gerber A L, et al. Genomic characterization of a novel SARS-CoV-2 lineage from Rio de Janeiro, Brazil. J Virol. 2021 Mar. 1. doi: 10.1128/JVI.00119-21
15. Singh J, Rahman S A, Ehtesham N Z, Hira S, Hasnain S E. SARS-CoV-2 variants of concern are emerging in India. Nat Med. 2021 July; 27(7):1131-3. doi:10.1038/s41591-021-01397-4
16. Wang L, Cheng G. Sequence analysis of the emerging SARS-CoV-2 variant Omicron in South Africa. J Med Virol. 2022 April; 94(4):1728-1733. doi: 10.1002/jmv.27516. Epub 2021 Dec. 27. PMID:34897752.
17. West A P, Wertheim J O, Wang J C, Vasylyeva T I, Havens J L, Chowdhury M A, et al. Detection and characterization of the SARS-CoV-2 lineage B.1.526 in New York. bioRxiv. doi:10.1101/2021.02.14.431043
18. Wink P L, Volpato F C Z, Monteiro F L, Willig J B, Zavascki A P, Barth A L, et al. First identification of SARS-CoV-2 Lambda (C.37) variant in Southern Brazil. medRxiv. doi:10.1101/2021.06.21.21259241
19. Younes A, Bartlett N L, Leonard J P, et al. Brentuximab vedotin (SGN-35) for relapsed CD30-positive lymphomas. N Engl J Med 2010; 363: 1812-21. Doi: 10.1056/NEJMoa1002965
20. Petersdorf S H, Kopecky K J, Slovak M, Willman C, Nevill T, Brandwein J, Larson R A, Erba H P, Stiff P J, Stuart R K, Walter R B, Tallman M S, Stenke L, Appelbaum F R. A phase 3 study of gemtuzumab ozogamicin during induction and postconsolidation therapy in younger patients with acute myeloid leukemia. Blood. 2013 Jun. 13; 121(24):4854-60. doi:10.1182/blood-2013-01-466706.
21. Olsen E, Duvic M, Frankel A, et al. Pivotal phase III trial of two dose levels of denileukin diftitox for the treatment of cutaneous T-cell lymphoma. J Clin Oncol 2001; 19:376-88. doi:10.1200/JCO.2001.19.2.376
22. Kreitman R J, Tallman M S, Robak T, et al. Phase I trial of anti-CD22 recombinant immunotoxin moxetumomab pasudotox (CAT-8015 or HA22) in patients with hairy cell leukemia. J Clin Oncol 2012; 30:1822-28. Doi:10.1200/JCO.2011.38.1756
23. Pruszynski M, Koumarianou E, Vaidyanathan G, Revets H, Devoogdt N, Lahoutte T, et al. Improved tumor targeting of anti-HER2 nanobody through N-Succinimidyl 4-Guanidinomethyl-3-iodobenzoate radiolabeling. J Nuclear Med 2014; 55:650-6. doi:10.2967/jnumed.113.127100

1. An antibody or antibody fragment binding a conserved region of the spike protein of a Sarbecovirus, having an antigen binding site comprising:
a) a heavy chain variable sequence comprising:
i) a CDR1 comprising an amino acid sequence selected from the group consisting of SEQ ID NO:1, SEQ ID NO:2, SEQ ID NO:3, SEQ ID NO:4 and SEQ ID NO:93;
ii) a CDR2 comprising an amino acid sequence selected from the group consisting of SEQ ID NO:5, SEQ ID NO:6, SEQ ID NO:7, SEQ ID NO:8 and SEQ ID NO:94;
iii) a CDR3 comprising an amino acid sequence selected from the group consisting of SEQ ID NO:9, SEQ ID NO:10, SEQ ID NO:11, SEQ ID NO:12 and SEQ ID NO:95;
and/or
b) a light chain variable sequence comprising:
i) a CDR1 comprising an amino acid sequence selected from the group consisting of SEQ ID NO:13, SEQ ID NO:14, SEQ ID NO:15, SEQ ID NO:16 and SEQ ID NO:96;
ii) a CDR2 comprising an amino acid sequence selected from the group consisting of GAS, AAS and DAS;
iii) a CDR3 comprising an amino acid sequence selected from the group consisting of SEQ ID NO:20, SEQ ID NO:21, SEQ ID NO:22, SEQ ID NO:23, SEQ ID NO:24 and SEQ ID NO:97.

2. The antibody or antibody fragment according to claim 1, wherein the heavy chain variable sequence further comprises:
1) a framework region (FR1) comprising an amino acid sequence selected from the group consisting of SEQ ID NO:25, SEQ ID NO:26, SEQ ID NO:27, SEQ ID NO:28 and SEQ ID NO:98;
2) a framework region (FR2) comprising an amino acid sequence selected from the group consisting of SEQ ID NO:29, SEQ ID NO:30, SEQ ID NO:31, SEQ ID NO:32 and SEQ ID NO:99;
3) a framework region (FR3) comprising an amino acid sequence selected from the group consisting of SEQ ID NO:33, SEQ ID NO:34, SEQ ID NO:35, SEQ ID NO:36 and SEQ ID NO:100;
4) a framework region (FR4) comprising an amino acid sequence selected from the group consisting of SEQ ID NO:37, SEQ ID NO:38, SEQ ID NO:39, SEQ ID NO:40 and SEQ ID NO:101;
and/or
wherein the light chain variable sequence further comprises:
5) a framework region (FR1) comprising an amino acid sequence selected from the group consisting of SEQ ID NO:41, SEQ ID NO:42, SEQ ID NO:43; SEQ ID NO:44, SEQ ID NO:45 and SEQ ID NO:102;
6) a framework region (FR2) comprising an amino acid sequence selected from the group consisting of SEQ ID NO:46, SEQ ID NO:47, SEQ ID NO:48, SEQ ID NO:49, SEQ ID NO:50 and SEQ ID NO:103;
7) a framework region (FR3) comprising an amino acid sequence selected from the group consisting of SEQ ID NO:51, SEQ ID NO:52, SEQ ID NO:53, SEQ ID NO:54, SEQ ID NO:55 and SEQ ID NO:104;
8) a framework region (FR4) comprising an amino acid sequence selected from the group consisting of SEQ ID NO:56, SEQ ID NO:57, SEQ ID NO:58, SEQ ID NO:59 and SEQ ID NO:105.

3. The antibody or antibody fragment according to claim 1 wherein the heavy chain variable sequence and/or light chain sequence comprises the following combination of CDRs:
a) CDR1 comprising the amino acid sequence SEQ ID NO:1; CDR2 comprising the amino acid sequence SEQ ID NO:5; CDR3 comprising the amino acid sequence SEQ ID NO:9 for the heavy chain variable sequence; CDR1 comprising the amino acid sequence SEQ ID NO:13; CDR2 comprising the amino acid sequence GAS; CDR3 comprising the amino acid sequence SEQ ID NO:20 for the light chain variable sequence;
b) CDR1 comprising the amino acid sequence SEQ ID NO:1; CDR2 comprising the amino acid sequence SEQ ID NO:5; CDR3 comprising the amino acid sequence SEQ ID NO:9 for the heavy chain variable sequence; CDR1 comprising the amino acid sequence SEQ ID NO:13; a CDR2 comprising or consisting of the amino acid sequence GAS; a CDR3 comprising the amino acid sequence SEQ ID NO:21 for the light chain variable sequence;
c) CDR1 comprising the amino acid sequence SEQ ID NO:2; CDR2 comprising the amino acid sequence SEQ ID NO:6; a CDR3 comprising the amino acid sequence SEQ ID NO:10 for the heavy chain variable sequence; CDR1 comprising the amino acid sequence SEQ ID NO:14; a CDR2 comprising the amino acid sequence AAS; a CDR3 comprising the amino acid sequence SEQ ID NO:22 for the light chain variable sequence;
d) CDR1 comprising the amino acid sequence SEQ ID NO:3; a CDR2 comprising the amino acid sequence SEQ ID NO:7; a CDR3 comprising the amino acid sequence SEQ ID NO:11 for the heavy chain variable sequence; CDR1 comprising the amino acid sequence SEQ ID NO:15; a CDR2 comprising the amino acid sequence AAS; a CDR3 comprising the amino acid sequence SEQ ID NO:23 for the light chain variable sequence;
e) CDR1 comprising the amino acid sequence SEQ ID NO:4; CDR2 comprising the amino acid sequence SEQ ID NO:8; CDR3 comprising the amino acid sequence SEQ ID NO:12 for the heavy chain variable sequence; CDR1 comprising the amino acid sequence SEQ ID NO:16; CDR2 comprising the amino acid sequence DAS; CDR3 comprising the amino acid sequence SEQ ID NO:24 for the light chain variable sequence;
f) CDR1 comprising the amino acid sequence SEQ ID NO:93; CDR2 comprising the amino acid sequence SEQ ID NO:94; CDR3 comprising the amino acid sequence SEQ ID NO:95 for the heavy chain variable sequence; CDR1 comprising the amino acid sequence SEQ ID NO:96; CDR2 comprising the amino acid sequence AAS; CDR3 comprising the amino acid sequence SEQ ID NO:97 for the light chain variable sequence.

4. The antibody or antibody fragment according to claim 3, wherein said heavy chain variable sequence and/or said light chain variable sequence further comprises the following combination of FRs:
a) FR1 comprising SEQ ID NO:25; FR2 comprising SEQ ID NO:29; FR3 comprising SEQ ID NO:33 and FR4 comprising SEQ ID NO:37 for the heavy chain variable sequence; FR1 comprising SEQ ID NO:41; FR2 comprising SEQ ID NO:46; FR3 comprising SEQ ID NO:51 and a framework region FR4 comprising SEQ ID NO:56 for the light chain variable sequence;
b) FR1 comprising SEQ ID NO:25; FR2 comprising SEQ ID NO:29; FR3 comprising SEQ ID NO:33 and FR4 comprising SEQ ID NO:38 for the heavy chain variable sequence; FR1 comprising SEQ ID NO:42; FR2 comprising SEQ ID NO:47; FR3 comprising SEQ ID NO:52, and FR4 comprising SEQ ID NO:57 for the light chain variable sequence;
c) FR1 comprising SEQ ID NO:26; FR2 comprising SEQ ID NO:30; FR3 comprising SEQ ID NO:34 and FR4 comprising SEQ ID NO:39 for the heavy chain variable sequence; FR1 comprising SEQ ID NO:43; FR2 comprising SEQ ID NO:48; FR3 comprising SEQ ID NO:53, and FR4 comprising SEQ ID NO:58 for the light chain variable sequence;
d) FR1 comprising SEQ ID NO:27; FR2 comprising SEQ ID NO:31; FR3 comprising SEQ ID NO:35 and FR4 comprising SEQ ID NO:40 for the heavy chain variable sequence; FR1 comprising SEQ ID NO:44; FR2 comprising SEQ ID NO:49; FR3 comprising SEQ ID NO:54, and FR4 comprising SEQ ID NO:56 for the light chain variable sequence;
e) FR1 comprising SEQ ID NO:28; FR2 comprising SEQ ID NO:32; FR3 comprising SEQ ID NO:36; FR4 comprising SEQ ID NO:39 for the heavy chain variable sequence; FR1 comprising SEQ ID NO:45; FR2 comprising SEQ ID NO:50; FR3 comprising SEQ ID NO:55 and a FR4 comprising SEQ ID NO:59 for the light chain variable sequence;
f) FR1 comprising SEQ ID NO:98; FR2 comprising SEQ ID NO:99; FR3 comprising SEQ ID NO:100; FR4 comprising SEQ ID NO:101 for the heavy chain variable sequence; FR1 comprising SEQ ID NO:102; FR2 comprising SEQ ID NO:103; FR3 comprising SEQ ID NO:104 and a FR4 comprising SEQ ID NO:105 for the light chain variable sequence.

5. The antibody or antibody fragment according to claim 1, which comprises:
a) a heavy chain variable sequence comprising the amino acid sequence SEQ ID NO:60 and/or a light chain variable sequence comprising the amino acid sequence SEQ ID NO:61;
or
b) a heavy chain variable sequence comprising the amino acid sequence SEQ ID NO: 62 and/or a light chain variable sequence comprising the amino acid sequence SEQ ID NO: 63;
c) a heavy chain variable sequence comprising the amino acid sequence SEQ ID NO:64 and/or a light chain variable sequence comprising the amino acid sequence SEQ ID NO:65;
d) a heavy chain variable sequence comprising the amino acid sequence SEQ ID NO:66 and/or a light chain variable sequence comprising the amino acid sequence SEQ ID NO:67;
e) a heavy chain variable sequence comprising the amino acid sequence SEQ ID NO:68 and/or a light chain variable sequence comprising the amino acid sequence SEQ ID NO:69;
f) a heavy chain variable sequence comprising the amino acid sequence SEQ ID NO:70 and/or a light chain variable sequence comprising the amino acid sequence SEQ ID NO:71.

6. The antibody or antibody fragment according to claim 1 which is a monoclonal antibody.

7. The antibody or antibody fragment according to claim 1 which is an IgG selected from the group consisting of IgG1, IgG2, IgG3, IgG4; a Fab fragment; a single chain antibody (scFv); a bispecific antibody; optionally conjugated with a drug or a label.

8. A nucleotide sequence encoding for the amino acid sequence of the heavy chain variable region and/or the light chain variable region of the antibody or antibody fragment according to claim 1.

9. An expression vector comprising at least one nucleotide sequence according to claim 8.

10. A host cell comprising the expression vector according to claim 9.

11. A hybridoma comprising at least one of a nucleotide sequence encoding for the amino acid sequence of the heavy chain variable region and/or the light chain variable region for the production of the antibody or antibody fragment according to claim 1.

12. A formulation for molecular/vector-based passive immunoprophylaxis comprising at least one of the nucleotide sequence or expression vector according to claim 9 together with one or more pharmaceutically acceptable excipients and/or adjuvants.

13. The antibody or antibody fragment according to claim 1, for the treatment and/or prophylaxis of Sarbecoviruses-mediated diseases in a subject, in particular of the Severe Acute Respiratory Syndrome mediated by SARS CoV-1 or SARS-CoV-2 and variants thereof.

14. The antibody or antibody fragment for the treatment and/or prophylaxis of Sarbecoviruses-mediated diseases in a subject according to claim 13, wherein said variants are selected from the group consisting of Alpha (B.1.1.7), Beta (B.1.351) Gamma (P.1), Delta (B.1.617.2), D614 G (Università di Pavia), and Omicron (B.1.1.529) variants of SARS-CoV-2.

15. The antibody or antibody fragment according to claim 13, wherein the subject to be treated is an immunocompromised patient, a patient with an history of cardiovascular and/or respiratory diseases, an elderly patient or a vaccine-hesitant subject.

16. The antibody or antibody fragment according to claim 13, wherein a first antibody or antibody fragment is used alone or in combination with a second antibody or antibody fragment directed against a different conserved region of the spike protein.

17. A pharmaceutical composition comprising at least one of the antibody or antibody fragment of claim 1 as the active ingredient, together with one or more pharmaceutically acceptable excipients and/or adjuvants.

18. The pharmaceutical composition according to claim 17, suitable for intravenous, intramuscular or subcutaneous administration.

19. A composition or kit of parts comprising a first antibody or antibody fragment that binds a targeted conserved region of the spike protein of SARS-CoV-1 or SARS-CoV-2 and a second antibody or antibody fragment that binds a different targeted conserved region of the spike protein of SARS-CoV-1 or SARS-CoV-2 with respect to the first antibody, for the simultaneous, separate or sequential administration in a subject affected by Sarbecoviruses-mediated diseases, in particular by the Severe Acute Respiratory Syndrome mediated by SARS-CoV-1 or SARS-CoV-2.

20. The antibody or antibody fragment according to claim 1 configured to detect the presence of a Sarbecovirus, preferably of SARS-CoV-1 or SARS-CoV-2 in a biological sample.
